SEQUENCE LISTING (2 sequences)

SEQ ID NO: 1 - protein, 451 aa, artificial
  chimeric antibody
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 215 aa, artificial
  chimeric antibody
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2006/0008415 A1
(43) Pub. Date: Jan. 12, 2006

(54) Stable liquid and lyophilized formulation of proteins

(75) Inventors: Elisabet A. Kaisheva, Belmont, CA (US); Supriya Gupta, Sunnyvale, CA (US); Weichang Zhou, Livermore, CA (US); Robert Weinkam, San Carlos, CA (US); Patrick Powers, Palo Alto, CA (US); Naichi Liu, San Jose, CA (US); Vanitha Ramakrishnan, Belmont, CA (US)

Correspondence Address: LEGAL DEPARTMENT;PROTEIN DESIGN LABS, INC., 34801 CAMPUS DRIVE, FREMONT, CA, 94555, US

(73) Assignee: Protein Design Labs, Inc., Fremont, CA (US)

(21) Appl. No.: 11/166,906

(22) Filed: Jun. 24, 2005

Related U.S. Application Data
(60) Provisional application No. 60/583,127, filed on Jun. 25, 2004.

(51) Int. Cl. A61K 51/00; A61K 39/395

(52) U.S. Cl. 424/1.49; 424/130.1

(57) ABSTRACT

The present invention is directed to stable protein derivatives, e.g., antibodies, antibody fragments or peptides, with at least one free thiol group coupled to N-acetyl-L-cysteine, N-ethyl-maleimide, or cysteine and the methods of making such derivatives. In addition, stable liquid pharmaceutical formulations comprising such proteins or their derivatives and stable lyophilized pharmaceutical formulations comprising such proteins are provided. The present invention is also directed to a method of making a stable Fab′ fragment of an antibody and a method of controlling vascularization in injured or cancerous tissue comprising applying to the injured tissue one or more doses of the pharmaceutical formulations.

### CROSS-REFERENCE TO RELATED APPLICATIONS

Pursuant to 35 U.S.C. § 119(e) and any other applicable statute or rule, the present application claims benefit of and priority to U.S. Ser. No. 60/583,127 entitled “Stable Liquid and Lyophilized Formulations of Proteins,” filed Jun. 25, 2004 by Kaisheva, Gupta, Zhou, Weinkam, Powers, and Liu.

### FIELD OF THE INVENTION

The present invention relates generally to the field of immunology and pharmaceutical formulations. In particular, it concerns stable liquid and lyophilized pharmaceutical formulations comprising a protein, such as an antibody or a fragment thereof or a peptide, having one or more thiol groups linked to a stabilizing molecule. The protein, e.g., antibody, typically has a free thiol group and additional stabilizing components or excipients.

### BACKGROUND OF THE INVENTION

Antibodies and polypeptides are among the most important therapeutic proteins in use today for treating a variety of diseases including, but not limited to cancer, autoimmune diseases, heart failure, and infectious diseases.

A typical need in cancer treatment is for a treatment that is specific to cancer tissue while not harming normal tissue. Therefore, the specificity of antibodies and antibody fragments, e.g., antigen-binding Fab fragments, is highly desirable, as they have a specificity that is not typically provided by other molecules.

For example, growing tumors are characterized by a high level of angiogenesis activity. Angiogenic vasculature has a number of up-regulated cell surface markers, e.g., integrins, that are optionally targeted, by a chemotherapeutic molecule, to destroy or inhibit tumor tissue and leave normal tissue unharmed. For example, a chemotherapeutic molecule is optionally attached to an antibody or antibody fragment that specifically binds to a tumor cell and leaves normal tissue unharmed.

Small peptides are also used in the treatment of cancer, e.g., melanoma. Peptides that bind to the proteoglycan NG2/HM, a melanoma associated antigen, expression of which increases the proliferative capacity of melanoma cells, can be used to target melanoma cells. See, e.g., U.S. Pat. No. 6,528,481, describing non-antibody peptides that selectively target angiogenic vasculature, e.g., in a tumor.

Another method of inhibiting tumor growth involves a compound that blocks the Protein C system. For example, an anti-Protein C or anti-activated Protein C antibody is optionally used to disrupt the Protein C pathway. This blocks natural anticoagulant pathways and leads to microvascular thrombosis in tumor capillaries. In this pathway, the inhibitory effect may need to be reversed quickly in the event that thrombotic complications occur at sites other than the tumor. Therefore, a Fab or Fab′ fragment that has a shorter half-life than a full-length antibody is preferable. See, e.g., U.S. Pat. No. 6,423,313, by Esmon.

A shorter half-life is also desirable in other treatments, e.g., when preventing blood clotting or coagulation during procedures such as angioplasty. For example, cardiovascular disease, a leading cause of death in the United States, is currently treated used anti-thrombic antibodies and polypeptides. Such medications include heparin, aspirin, integrilin (a cyclic heptapeptide), anti-GP-IIb/IIIa antibodies, and the like. Typically, a short half-life is desirable in these medications so that the effect can be reversed or terminated if too much bleeding occurs. Antibody Fab′ fragments and small peptides are therefore useful for such treatments because they have a shorter half-life than full-length proteins or antibodies.

Naturally occurring antibodies (immunoglobulins) comprise two heavy chains linked together by disulfide bonds and two light chains, each light chain being linked to one of the heavy chains by disulfide bonds. Each chain has an N-terminal variable domain (VH or VL) and a constant domain at its C-terminus. The constant domain of the light chain is aligned with and disulfide bonded to the first constant domain of the heavy chain, and the light chain variable domain is aligned with the variable domain of the heavy chain. The heavy chain constant region includes (in the N- to C-terminal direction) the CH1, hinge, CH2 and CH3 regions.

Antibodies can be divided or fragmented into a variety of antigen-binding fragments. Papain digestion of most antibody molecules produces two Fab fragments containing the variable domain and the constant domain of the light chain dimerized with the variable domain and the first constant domain (CH1) of the heavy chain and a residual Fc domain. Each Fab fragment typically comprises a single antigen-binding fragment.

Fab′ fragments differ from Fab fragments in that they include a few additional residues at the carboxy terminus of the heavy chain CH1 domain including one or more cysteines from the antibody hinge region. Fab′-SH is the designation used herein for a Fab′ fragment in which the cysteine residue(s) of the constant domains contain a free thiol group. F(ab′)₂ antibody fragments produced by digestion of antibodies with papain, originally are produced as pairs of Fab′-SH fragments which are disulfide bonded via the hinge cysteines. As described below, Fab′-SH fragments are typically generated by papain digestion of antibodies, e.g., under certain circumstances. Due to the presence of an exposed free thiol group, however, the Fab′-SH fragments typically are not stable in liquid formulations.

In fact, many protein and peptide preparations intended for human use require stabilizers to prevent denaturation, aggregation and other alterations to the protein prior to using the preparation. This is a particular problem with proteins containing one or more free thiol groups because such molecules are especially prone to oxidation and aggregation.

Oxidation of cysteine residues in a protein results in the formation of both intra-and intermolecular disulfide bonds and can give rise to disulfide linked protein aggregates (see e.g., J. Biol. Chem. 267:11307-11315 (1992); Free Radical Biol. Med. 7:659-673(1989)). Oxidation of cysteine also results in the production of reactive oxygen species that can cause further oxidative damage to disulfide bonds as well as to other residues in the protein.

Some strategies employed to inhibit cysteine oxidation in liquid formulations include the use of metal chelators such as EDTA that makes metal ions unavailable to initiate the oxidation process (see e.g., Pharm. Res. 10:649-659(1993)). Other commonly used pharmaceutical antioxidants may also inhibit cysteine oxidation (see e.g., Biotechnol. Appl. Biochem. (2000) 32, 145-153; Adami, M et al., International Patent Application No. WO 92/01442). Cysteine oxidation can also be reduced by lowering the pH of the protein containing solution thereby protonating sulfhydryl groups (pKa 8.5) which inhibits their reaction with metal ions that initiate the oxidation reaction (see e.g., Biophys. J. 68:2218-2223(1995)).

Addition of excipients that serve as mild reducing agents, for example, cysteine, is also optionally used to reduce disulfide linked aggregate formation, e.g., resulting from oxidation of cysteines in the protein molecule. However, this approach has limited applicability in the development of liquid protein containing formulations because mixed disulfide bonds are often formed between the reactive reducing agent and the free thiol residues in the protein. Use of cysteine as a mild reducing agent to prevent aggregation is further limited due to the possible oxidation of free cysteine to form cystine, which has very low water solubility, and tends to precipitate over time.

Another existing approach is to make stable derivatives of the proteins and then formulate the derivatives in appropriate pharmaceutical solutions. In one example, the thiol groups are attached to a hydrophilic polymer (U.S. Pat. No. 6,210,707), or linked to hydrazine (U.S. Pat. No. 6,576,746) to form stable derivatives. Antibody fragments containing free thiol groups, such as Fab′ fragments are stabilized by being linked to polyethylene glycol (PEG) molecules, e.g, PEGylated antibodies, (see e.g., Chapman, A. P., et al, Advanced Drug Delivery Reviews 54: 531 -545 (2002)). Free thiol groups are also optionally stabilized through nitrosylation and/or s-nitrosation (see e.g., Sumbayev V. V. et al, FEBS Letters: 535: 106-112 (2003)).

Given the limited options available to stabilize proteins with reactive free thiols in a liquid formulations, other options for stabilization, such as lyophilization, are found in the literature (see e.g., “Formulation, Characterization, and Stability of Protein Drugs, Case Histories,” Eds. Rodney Pearlman and Y. John Wang, Pharmaceutical Biotechnology, Volume 9, Plemum Press, 1996, NY). However, additional stabilization methods are still needed for biological pharmaceuticals.

Given the importance of peptide and antibody pharmaceuticals and the limited options available to stabilize proteins with free thiols, e.g., in liquid formulations, a clear need for additional agents and methods for stabilizing those proteins remains. See, e.g., U.S. Pat. No. 6,475,488, describing fibronectin binding polypeptides for the inhibition of angiogenesis, which asserts that a need exists for protein pharmaceuticals of increased biological stability. The present invention fulfills these needs and others as described in detail below.

### SUMMARY OF THE INVENTION

The present invention provides stable liquid and lyophilized protein compositions and methods of preparing such compositions. For example, proteins comprising a free thiol group are coupled to sulfhydryl reactive molecules, e.g., N-acetyl-L-cysteine, N-ethyl-maleimide, or cysteine, to stabilize the protein, e.g., in a liquid formulation.

In one aspect, the present invention provides compositions comprising a protein, wherein the protein comprises a thiol group coupled to N-acetyl-L-cysteine, N-ethyl-maleimide, or cysteine. In one embodiment, the protein comprises an antibody or an antibody fragment, e.g., a Fab′ fragment. Typical antibodies of the invention comprise Fab′ fragments of IgG4 antibodies. In other embodiments, the proteins of the invention comprise antibodies that bind to integrins, e.g., α5β1 or α4β1 integrin, or anticoagulation proteins or peptides, e.g., Reopro®, Integrilin, or the like, and peptides used for the treatment of heart failure, e.g., urodilatin, nesiritide, and the like. In one embodiment, the present invention comprises an anti-α5β1 integrin antibody having the amino acid sequence of SEQ ID NOs: 1 and/or 2, or a Fab′ fragment thereof.

In another aspect, the present invention provides stable liquid or lyophilized pharmaceutical formulations comprising a protein or protein derivative and a pharmaceutically acceptable carrier, wherein the protein comprises a thiol group coupled to N-acetyl-L-cysteine, N-ethyl-maleimide, or cysteine. Typical proteins of the invention include, but are not limited to, antibodies, e.g., IgG4 antibodies, antibody fragments, e.g., Fab′ fragments, anti-coagulation proteins and peptides, and the like. For example, one pharmaceutical formulation of the invention comprises an antibody fragment that binds to α5β1 integrin, e.g., the antibody having the heavy chain amino acid sequence provided in SEQ ID NO: 1 and the light chain amino acid sequence of SEQ ID NO: 2.

In another aspect, the present invention provides methods for preparing protein compositions e.g., proteins that are coupled to a stabilizing agent, e.g., N-acetyl-L-cysteine, N-ethyl-maleimide, or cysteine. The methods typically comprise incubating a protein of the invention, e.g., an antibody or anti-coagulation peptide with a free thiol group, with N-acetyl-L-cysteine, N-ethyl-maleimide, or cysteine, e.g., in the presence of sodium tetrathionate, thereby coupling the stabilizing agent to the thiol group of the protein.

For example, the present invention provides methods of coupling a Fab′ fragment of an antibody to N-acetyl-L-cysteine. A typical method of the invention comprises digesting the antibody with papain, to produce a Fab′ fragment, wherein the Fab′ fragment comprises a free thiol group. The Fab′ fragment is then typically incubated with N-acetyl-cysteine in the presence of sodium tetrathionate, thereby coupling the N-acetyl-cysteine to the Fab′ fragment via the free thiol group. Additional steps, e.g., purifying the Fab′ fragment, are also provided herein.

### BRIEF DESCRIPTION OF THE DRAWING

FIG. 1 depicts a schematic of the papain digestion of M200 (an antibody having a heavy chain amino acid sequence of SEQ ID NO: 1 and a light chain amino acid sequence of SEQ ID NO: 2, or conservatively modified variations thereof) antibody to produce a Fab′ fragment, F200, with an exposed free thiol.

### DETAILED DESCRIPTIONS OF THE PREFERRED EMBODIMENTS

To address the problem of stability of proteins having free thiols such as Fab′-SH antibody fragments in liquid and lyophilized formulations, the present invention utilizes a stabilizing agent, e.g., a sulfhydryl reactive stabilizing molecule, for coupling to free thiols. The present invention therefore provides stabilized protein derivatives, e.g., for use in pharmaceuticals, and methods of making stabilized protein derivatives.

Preferred proteins of the invention include, but are not limited to, antibodies, antibody fragments, and peptides. The molecules of the invention are typically stabilized by coupling a free thiol in the molecule of interest to a stabilizing agent such as an N-acetyl-L-cysteine (NAC) molecule, a cysteine (CYS) molecule, or a N-ethylmaleimide (NEM) molecule. The free thiol is optionally at the terminus of a protein molecule and includes those that are internal to the polypeptide chain and those that are buried in the hydrophobic core of the protein molecule. Those that are buried in the core of the protein are partially unfolded, e.g., with denaturants such as urea or guanidine hydrochloride to expose the buried thiol for coupling to the stabilizing agent.

In a preferred embodiment, the proteins are IgG4 antibodies and more preferably are chimeric or humanized antibodies or fragments thereof. For example, the protein is optionally an antibody that binds to an integrin, e.g., α5β1 integrin, α4β1 integrin, or the like, or a Fab′-SH fragment of such antibodies. In other embodiments, the proteins are peptides, such as urodilatin, nesiritide, integrilin, and the like.

The stabilizing agents of the present invention include, but are not limited to, N-acetyl-L-cysteine (NAC), cysteine (CYS), and N-ethylmaleimide (NEM), or other sulfbydryl reactive molecules to which the proteins of the invention are coupled, e.g., via a disulfide bond. N-acetyl-L-cysteine (NAC), for example, is a molecule commonly used as an additive in food. It is a potent antioxidant and an approved inactive ingredient for nonparenteral administration to patients, such as in the form of tablets, capsules, powders, granules, or suspensions in non-aqueous solutions (see e.g., U.S. Pat. Nos. 4,920,122; 6,207,190; and 6,689,385). Waterman K., et al also disclose the use of NAC as an anti-oxidant in both liquid and solid formulations (Waterman K., et al, Pharmaceutical Development and Technology 7(1): 1-32 (2002)).

According to the present invention, NAC, NEM, and/or CYS are also optionally used as excipients to stabilize proteins in liquid or lyophilized formulations without coupling to free thiols. This approach allows the stabilization of the protein having a free thiol in the liquid formulation prior to the start of the lyophilization process, and also in the lyophilized product by reducing or inhibiting the formation of the disulfide-linked aggregates. The methods and compositions of the invention are described in more detail below.

As used herein, the phrase “protein derivative” refers to a protein having a thiol group coupled to NAC, NEM, CYS or other sulfhydryl reactive molecules. “Protein” as used herein includes, but is not limited to, proteins, antibodies, antibody fragments, polypeptides, peptides, and the like. For example, a peptide off the invention is typically about 5 to about 50 amino acids. Furthermore, the proteins of the invention are optionally naturally occurring proteins or non-naturally occurring proteins.

The term “pharmaceutical formulation” refers to physiologically acceptable excipients and carrier solutions well known to those of ordinary skill in the art. Methods for developing suitable dosing and treating regimens for using the particular pharmaceutical formulations are also well known to those of ordinary skill in the art. The pharmaceutical formulations of the present invention allow the proteins or protein derivatives to remain physically, chemically and biologically stable.

“Stable” (or “stability”) as used in the context of the present invention means that the protein composition retains its physical stability and/or chemical stability and/or biological activity upon storage. Various analytical techniques for measuring protein stability for predetermined times and temperatures stability are well known in the art and are reviewed in e.g., “Peptide and Protein Drug Delivery,” 247-301, Vincent Lee Ed., Marcel Dekker, Inc., New York, N.Y., Pubs. (1991) and Jones, A. Adv. Drug Delivery Rev. 10:29-90 (1993). Stability is optionally measured, for example, after exposure to a selected temperature for a selected time period.

A protein, e.g., an antibody, antibody fragment, polypeptide, or peptide, “retains its physical stability” in a pharmaceutical formulation if it shows no significant increase in aggregation, precipitation and/or denaturation, e.g., upon visual examination of color and/or clarity, or as measured by UV light scattering, size exclusion chromatography (SEC), SDS-PAGE or other methods well known in the art. Protein denaturation is also optionally evaluated by fluorescence to determine the tertiary structure, by circular dichroism spectroscopy (CD spectroscopy) that measures changes in secondary and tertiary structures, and/or by FTIR to determine the secondary structure.

A protein, e.g., an antibody, antibody fragment, or polypeptide, “retains its chemical stability”, e.g., in a pharmaceutical formulation, if it shows no significant chemical alteration. Chemical stability is optionally assessed by detecting and/or quantifying chemically altered forms of the protein. Chemical alteration optionally involves size modification (e.g. clips or clipping) that is typically evaluated using size exclusion chromatography, SDS-PAGE and/or matrix-assisted laser desorption ionization/time-of-flight mass spectrometry (MALDI/TOF MS) of other analytical methods well known to one of ordinary skill in the art. Other types of chemical alteration include charge alteration (e.g. occurring as a result of deamidation) which can be evaluated by ion-exchange chromatography. Clipping/deamidation and/or isomerization may result in change in the CIEF profile. Deamidation and/or isomerization may also result in iso-aspartic acid formation, which is readily determined by well-known methods in the art.

A protein, e.g., an antibody, antibody fragment, polypeptide, or peptide, “retains its biological activity” in a pharmaceutical formulation, if the biological activity of the protein at a given time is within a predetermined range of the biological activity exhibited at the time the pharmaceutical formulation was prepared. Where the protein is an antibody, the biological activity of an antibody is optionally determined, for example, by an antigen-binding assay.

A “stable liquid formulation” or “stable lyophilized formulation” comprises a liquid formulation or lyophilized formulation comprising a protein, e.g., an antibody or fragment thereof or protein derivative as described herein, that exhibits no significant physical, chemical, or biological changes in the protein when stored at a refrigerated temperature, e.g., about 2° C. to about 8° C., for at least about 12 months, preferably about 2 years, and more preferably about 3 years; or at room temperature, e.g., about 22° C. to about 28° C., for at least about 3 months, preferably about 6 months, and more preferably about 1 year. The criteria for stability are as follows: no more than about 10%, and preferably no more than about 5%, of protein monomer is degraded as measured by SEC-HPLC. Preferably, the solution remains colorless, or clear to slightly opalescent by visual analysis. The concentration, pH and osmolality of the formulation have no more than about ±10% change. Potency is typically within about 70-130%, and preferably 80-120% of a control level. No more than about 10%, and preferably no more than about 5% clipping of the protein is observed. No more than about 10%, and preferably no more than about 5% of protein forms aggregates.

The term “buffer” encompasses those agents which maintain the pH value of a solution, e.g., in an acceptable range and includes, but is not limited to, sodium citrate, succinate (sodium or potassium), histidine, phosphate (sodium or potassium), TRIS® (tris(hydroxymethyl) aminomethane), diethanolamine, and the like. A preferred buffer has a pH in the range from about 5.0 to about 8.0; and preferably has a pH of about 6.0 to 7.0. Examples of buffers that will control pH in this range include succinate (such as sodium succinate), gluconate, histidine, citrate, phospate and other organic acid buffers.

The terms “lyophilized,” and “freeze-dried” refer to a material that is first in a “pre-lyophilized” liquid form and which is subsequently frozen and sublimed in a vacuum environment to remove the ice or frozen solvent. During the lyophilization process an excipient is optionally included in the pre-lyophilized liquid formulation, e.g., to enhance the stability of the lyophilized product upon storage.

The term “bulking agent” includes agents that can provide additional structure to a freeze-dried product (e.g., to provide a pharmaceutically acceptable cake). Commonly used bulking agents include mannitol, glycine, lactose, sucrose, and the like. In addition to providing a pharmaceutically acceptable cake, bulking agents also typically impart useful qualities to the lyophilized composition such as modifying the collapse temperature, providing freeze-thaw protection, further enhancing the protein stability over long-term storage, and the like. These agents can also serve as tonicity modifiers.

The term “cryoprotectants” generally includes agents that stabilize the protein or protein derivative against freezing-induced stresses. They also typically offer protection during primary and secondary drying, and long-term product storage. Examples of such cryoprotectants are polymers such as dextran and polyethylene glycol; sugars such as sucrose, glucose, trehalose, and lactose; surfactants such as polysorbates; and amino acids such as glycine, arginine, serine, and the like.

The term “lyphoprotectant” includes agents that provide stability to a protein during a drying or ‘dehydration’ process (primary and secondary drying cycles), presumably by providing an amorphous glassy matrix and by binding with the protein or protein derivative through hydrogen bonding, e.g., replacing the water molecules that are removed during the drying process. This helps to maintain protein conformation, minimize protein degradation during a lyophilization cycle, and improve the long-term stability of the protein or protein derivative. Examples include polyols or sugars such as sucrose and trehalose.

“Reconstitution time” is the time that is required to rehydrate a lyophilized formulation with a liquid, e.g., to provide a particle-free clarified solution.

The term “isotonic” means that the formulation of interest has essentially the same osmolarity as human blood. Isotonic formulations generally have an osmolarity of about 270-328 mOsm. Slightly hypotonic osmolarity in pressure is about250-269 mOsm and slightly hypertonic is about 328-350 mOsm. Osmolarity is measured, for example, using a vapor pressure or ice-freezing type osmometer.

Tonicity modifiers useful in the formulations of the present invention include, for example, salts, e.g., NaCl, KCl, MgCl₂, CaCl₂, and the like, and are used to control osmolarity. In addition, cryprotecants/lyoprotectants and/or bulking agents such as sucrose, mannitol, glycine, and others can serve as tonicity modifiers.

I. Proteins and Methods for Producing Them

A protein is a polymer of amino acid residues. In the present invention, the term “protein” encompasses naturally occurring amino acids and polymers thereof as well as amino acid polymers in which one or more amino acid residues is an artificial chemical mimetic of a naturally occurring amino acid, as well as amino acid polymers containing modified residues, and non-naturally occurring amino acid polymers.

Amino acids include naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs include compounds that have the same basic chemical structure as a naturally occurring amino acid, e.g., an α carbon that is bound to a hydrogen, a carboxyl group, an amino group, and an R group. Such analogs include, but are not limited to, homoserine, norleucine, methionine sulfoxide, methionine methyl sulfonium. Such analogs optionally include modified R groups (e.g., norleucine) or modified peptide backbones, but retain the same basic chemical structure as a naturally occurring amino acid. “Amino acid mimetic” refers to a chemical compound that has a structure that is different from the general chemical structure of an amino acid, but functions similarly to a naturally occurring amino acid.

Proteins encompassed by the present invention include all types of proteins including secreted proteins, transmembrane proteins or intracellular proteins. Preferred proteins comprise antibodies or fragments thereof or peptides, e.g., for use in the treatment of cancer or heart failure.

Currently available antibody pharmaceuticals that can benefit from the stabilizing methods and compositions provided herein include, but are not limited to, trastuzumab, (Herceptin®, Genentech, Inc); omalizumab, (Xolair®) efalizumab (Raptiva™, Genentech, Inc); bevacizumab (Avastin™, Genentech, Inc); daclizumab (Zenapax®, Roche); palivizumab (Synagis®, MedImmune, Inc); natalizumab (Tysabri®), alemtuzumab (Campath®, cetuximab (Erbitux®), infliximab (Remicade®), rituximab (Rituxan®), basiliximab (Simulect®), palivizumab (Synagis®), and gemtuzumab ozogamicin (Mylotarg®, Wyeth). In addition, the following therapeutic products, which are in various stages of development, are also optionally used in the methods and compositions of the invention: epratuzumab, (Vitaxin®), apolizumab (Zamyl®), and labetuzuma (CEA-Cide®).

Additional preferred proteins of the invention comprise polypeptides, e.g., anti-coagulant polypeptides as described in, e.g., U.S. Pat. No. 6,239,101 (Esmon et al.). For example, Eptifibatide (Integrelin®) is an intravenous cyclical heptapeptide that selectively blocks the platelet glycoprotein IIb/IIIa receptor. It reversibly binds to platelets and has a short half-life. It has demonstrated efficacy in the treatment of patients during coronary angioplasty, myocardial infarction and angina.

Abciximab (Reopro® Centocor B.V.) is the Fab fragment of the chimeric human-murine monoclonal antibody 7E3. This antibody binds to glycoprotein IIb/IIIa receptor of human platelets and inhibits platelet aggregation. It also binds to a vitronection αvβ3 receptor on platelets. Reopro® is multi-receptor antagonist that reduces complications associated with coronary angioplasty by preventing the formation of blood clots by inhibiting platelet aggregation.

A natural human peptide called human B-type natriuretic peptide (hBNP) that is secreted by the heart as part of the body's normal response to heart failure is the basis for another peptide pharmaceutical, e.g., Natrecor® (nesiritide), a recombinant form of the endogenous human peptide. Natrecor® is used in the treatment of acute heart failure.

Listed above are various peptides, polypeptides, and antibodies that are optionally stabilized using the methods described herein. It will be apparent to one skilled in the art upon review of the following detailed description that many other proteins are optionally stabilized using the compositions and methods provided herein.

Naturally occurring proteins of the present invention can be isolated and purified with the methods well known in the art, for example, hydroxylapatite chromatography, gel electrophoresis, dialysis, and affinity chromatography, with affinity chromatography being the preferred purification technique. Other purification techniques such as fractionation on an ion-exchange column, ethanol precipitation, reverse phase HPLC, chromatography on silica, chromatography on heparin SEPHAROSET™ chromatography on an anion or cation exchange resin (such as a polyaspartic acid column), chromatofocusing, SDS-PAGE, and ammonium sulfate precipitation are also available.

Proteins of the present invention are also optionally produced recombinantly. DNA molecules encoding the proteins of the present invention are used together with a variety of expression vectors to express the proteins, for example, in prokaryotic or eukaryotic cells. Expression vectors and recombinant DNA technology are well known to those of skill in the art (see, e.g., Ausubel, supra, and Gene Expression Systems (Fernandez & Hoeffler, eds, 1999)). The proteins of the present invention are typically produced by culturing a host cell transformed with an expression vector containing nucleic acid encoding the proteinof interest, e.g, an anti-coagulant peptide, under appropriate conditions to induce or cause expression of the protein. Conditions appropriate for protein expression will vary with the choice of the expression vector and the host cell, and are easily ascertained by one skilled in the art through routine experimentation or optimization. Appropriate host cells include yeast, bacteria, archaebacteria, fungi, insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), NS0 cells, THP1 cells (a macrophage cell line) and various other human cells and cell lines. The recombinantly produced proteins are also optionally purified, e.g., by any techniques discussed above or known in the art.

In a preferred embodiment, proteins of the present invention contain one or more thiol groups, which can be located in any domain or region of the protein. In one aspect, the thiol groups are exposed, i.e., on the surface of protein so that they may react, e.g., with NAC, NEM or CYS. In another aspect of the invention, the thiol groups are hidden, e.g., buried within any folded three-dimensional structures of the protein. In that case, the proteins are partially unfolded with denaturants such as urea or guanidine hydrochloride, e.g., to make the hidden thiol group available to react with NAC, NEM or CYS, or the like. The denaturant is then typically removed, e.g., to allow the protein, such as an anti-integrin antibody, to refold back to its active (or native) three-dimensional structure.

II. Antibodies and Methods for Producing Them

A typical protein that is stabilized according to the present invention comprises an antibody. For the purpose of the present invention, the term “antibody” includes an immunoglobulin molecule immunologically reactive with a particular antigen, and includes both polyclonal and monoclonal antibodies. The term also includes genetically engineered forms such as humanized (e.g., humanized murine antibodies), primatized or chimeric antibodies and heteroconjugate antibodies (e.g., bispecific antibodies). The term “antibody” also encompasses antigen binding forms or parts of antibodies, including fragments with antigen-binding capability (e.g., Fab′, Fab′-SH, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). In addition, the term “antibody” also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol :5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen (i.e., that binds to the antigen) can be generated by recombinant methods such as selection from libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin comprises a heavy and light chain. Each heavy and light chain contains a constant region and a variable region, (the regions are also referred to as “domains”). Light and heavy chain variable regions contain four “framework” regions interrupted by three hypervariable regions, also called “complementarity-determining regions” or “CDRs”. Sequences of the framework regions of different light or heavy chains are relatively conserved within a species. The framework region of an antibody, typically the combined framework regions of the constituent light and heavy chains, serves to position and align the CDRs in three-dimensional space.

The term “V_{H}” refers to the variable region of an immunoglobulin heavy chain of an antibody, including the heavy chain of an Fv, scFv, Fab′-SH or Fab. References to “V_{L}” refer to the variable region of an immunoglobulin light chain, including the light chain of an Fv, scFv, dsFv, Fab′-SH or Fab.

CDRs are primarily responsible for binding of an antibody or fragment thereof to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The phrase “single chain Fv” or “scFv” refers to an antibody in which the variable domains of the heavy chain and of the light chain of a traditional two chain antibody have been joined to form one polypeptide chain. Typically, a linker peptide is inserted between the two chains to allow for proper folding and creation of an active antigen binding site.

An antibody of the invention, e.g., an anti-integrin antibody, is optionally a chimeric antibody. A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. In a preferred embodiment, the variable regions of the chimeric antibody are derived from mouse, while the constant regions are derived from human. In order to produce the chimeric antibodies, the portions derived from two different species (e.g., human constant region and murine variable or binding region) can be joined together chemically by conventional techniques or can be prepared as single contiguous proteins with genetic engineering techniques. The DNA molecules encoding the proteins of both the light chain and heavy chain portions of the chimeric antibody can be expressed as contiguous proteins. The method of making the chimeric antibody is disclosed in U.S. Pat. No. 5,677,427; U.S. Pat. No. 6,120,767; and U.S. Pat. No. 6,329,508, each of which is incorporated by reference in its entirety.

A preferred antibody of the present invention is a humanized antibody. A “humanized antibody” is an immunoglobulin molecule that contains minimal sequence derived from non-human immunoglobulin. Humanized antibodies include human immunoglobulins (recipient antibody) in which its native CDRs are replaced by residues from a CDR of a non-human species (donor antibody) such as mouse, rat, rabbit, or the like, having the desired specificity, affinity and capacity. In some instances, corresponding non-human residues replace Fv framework residues of the human immunoglobulin. Humanized antibodies also optionally comprise residues that are found neither in the recipient antibody nor in the imported CDR or framework sequences. Typically, a humanized antibody comprises substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody will optimally also comprise at least a portion of an immunoglobulin constant region (Fc), typically that of a human immunoglobulin. For humanization methods and antibodies, see, Queen et al., U.S. Pat. Nos. 5,530,101; 5,585,089; 5,693,762; and 6,180,370 (each of which is incorporated by reference in its entirety). See also, Jones et al., Nature 321:522-525 (1986); Riechmann et al., Nature 332:323-329 (1988); and Presta, Curr. Op. Struct. Biol. 2:593-596 (1992)). Humanization of antibodies can also be performed following the methods of e.g. Winter and co-workers (Jones et al., Nature 321:522-525 (1986); Riechmann et al., Nature 332:323-327 (1988); or Verhoeyen et al., Science 239:1534-1536 (1988)), by substituting rodent CDRs or CDR sequences for the corresponding sequences of a human antibody. See also, U.S. Pat. No. 5,585,089.

Antibodies useful in the practice of the present invention are also optionally fully human antibodies. Fully human antibodies are optionally produced by a variety of techniques. One example is trioma methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference herein in its entirety). Fully human antibodies are also optionally produced from non-human transgenic animals having transgenes encoding at least a segment of the human immunoglobulin locus. The production and properties of animals having these properties are described in detail by, e.g., Lonberg et al., WO 93/12227; U.S. Pat. No. 5,545,806; and Kucherlapati, et al., WO 91/10741; U.S. Pat. No. 6,150,584, each of which is incorporated herein by reference in its entirety.

Various recombinant antibody library technologies are also optionally utilized to produce fully human antibodies. For example, one approach is to screen a DNA library from human B cells according to the general protocol outlined by Huse et al., Science 246:1275-1281 (1989). Antibodies or fragments thereof are selected from this library, typically by binding to a preselected antigen or a fragment thereof. Sequences encoding such antibodies (or binding fragments of an antibody) are then cloned and amplified. The protocol described by Huse is rendered more efficient in combination with phage-display technology. See, e.g., Dower et al., WO 91/17271 and McCafferty et al., WO 92/01047; U.S. Pat. No. 5,969,108, (each of which is incorporated by reference in its entirety). In these methods, libraries of phage are produced in which members display different antibodies on their outer surfaces. Antibodies are usually displayed as Fv, scFv or Fab′-SH fragments. Phage displaying antibodies with a desired specificity are selected by binding to the antigen or fragment thereof.

Eukaryotic ribosomes are optionally used as means to display a library of antibodies and which may be selected by screening against a target antigen, such as α5β1, as described in Coia G, et al., J. Immunol. Methods 1: 254 (1-2):191-7 (2001); Hanes J. et al., Nat. Biotechnol. 18 (12):1287-92 (2000); Proc. Natl. Acad. Sci. U.S.A. 95 (24):14130-5 (1998); Proc. Natl. Acad. Sci. U.S.A. 94 (10):4937-42 (1997), each of which is incorporated by reference in its entirety.

Antibody libraries are also optionally displayed on the surface of yeast cells for the purpose of obtaining the human antibodies and their encoding nucleic acid against a target antigen. This method is described by Yeung, et al., Biotechnol. Prog. 18(2):212-20 (2002); Boeder, E. T., et al., Nat. Biotechnol. 15(6):553-7 (1997), each of which is herein incorporated by reference in its entirety. Alternatively, human antibody libraries are expressed intracellularly and screened via yeast two-hybrid system (WO0200729A2, which is incorporated by reference in its entirety).

The antibodies of the present invention are optionally further purified, e.g., using, hydroxylapatite chromatography, gel electrophoresis, dialysis, and affinity chromatography, with affinity chromatography, e.g., using protein A, being the preferred purification technique. The suitability of protein A as an affinity ligand typically depends on the species and isotype of any immunoglobulin Fc domain that is present in the antibody. Protein A is optionally used to purify antibodies that are based on human Υ₁, Υ₂, or Υ₄ heavy chains (Lindmark et al., J. Immunol. Meth. 62:1-13 (1983)). Protein G is recommended as an affinity ligand for all mouse isotypes and for human Υ₃ (Guss et al., EMBO J. 5:1567-1575 (1986)). The matrix to which the affinity ligand is attached is typically agarose, but other matrices are optionally used. For example, mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSET™ chromatography on an anion or cation exchange resin (such as a polyaspartic acid column), chromatofocusing, SDS-PAGE, and ammonium sulfate precipitation are also available depending on the antibody to be recovered.

Antibodies of the present invention are typically derived from species including, but not limited to, human, chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety).

The antibodies of the present invention include antibodies having all types of constant regions, including IgM, IgG, IgD, IgA and IgE, and any isotype, including IgG1, IgG2a, IgG2b, IgG3 and IgG4, with IgG4 as a preferred isotype. The light chains of the antibodies are optionally either kappa light chains or lambda light chains. The antibodies typically bind to their epitopes at a binding affinity of at least 10⁶M⁻¹, 10⁷M⁻¹, 10⁸M⁻¹, 10⁹M⁻¹, or 10¹⁰M⁻¹.

In a preferred embodiment, the antibodies or antibody fragment of the present invention are antibodies against α5β1 integrin which bind specifically to at least one subunit of α5β1 integrin. The binding specificity of antibodies is optionally assessed by the methods known in the art such as concurrent immunoelectrophoresis, radioimmuno-assays, radioimmuno-precipitation, enzyme-linked immuno-sorbent assays (ELISA), dot blot or Western blot assays, inhibition or competition assays, and sandwich assays. For a review of immunological and immunoassay procedures, see, e.g., Basic and Clinical Immunology (Stites & Terr eds., 7^{th} ed. 1991).

Antibodies of the invention are optionally provided in a variety of forms, such as monoclonal, polyclonal, chimeric, humanized, fully human, and/or bispecific antibodies, e.g., against α5β1 integrin or fragments thereof. These antibodies are typically made by any method known in the art and/or discussed above.

The anti-α5β1 integrin antibodies of the present invention preferably neutralize at least one biological activity of an α5β1 integrin, such as receptor binding activity, signaling transduction, and cellular responses induced by α5β1. Preferably, such neutralizing antibodies are capable of competing with the binding of α5β1 to its signaling molecules, or even block the binding completely. Such antibodies preferably inhibit tumor angiogenesis and/or induce death of the proliferating endothelial cells.

In a preferred embodiment, the anti-α5β1 integrin antibodies are those disclosed in U.S. patent application Ser. No. 10/724,274, filed Nov. 26, 2003, (Publication No.: US 2005/0054834 A1,which is incorporated by reference in its entirety), which discloses the anti-α5β1 integrin antibody M200, which is a high affinity chimeric IgG4 antibody (with a human IgG4 constant region). M200 comprises a heavy chain an amino acid sequence as follows:

[SEQ ID NO: 1]

QVQLKESGPGLVAPSQSLSITCTISGFSLTDYGVHWVRQPPGKGLEWLVV
IWSDGSSTYNSALKSRMTIRKDNSKSQVFLIMNSLQTDDSAMYYCARHGT
YYGMTTTGDALDYWGQGTSVTVSSASTKGPSVFPLAPCSRSTSESTAALG
CLVKDYFPEPVTVSWNSGALTSGVHTFPAVLQSSGLYSLSSVVTVPSSSL
GTKTYTCNVDHKPSNTKVDKRVESKYGPPCPSCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLG
K.

M200 also comprises a light chain amino acid sequence as follows:

[SEQ ID NO: 2]

QIVLTQSPAIMSASLGERVTMTCTASSSVSSNYLHWYQQKPGSAPNLWIY
STSNLASGVPARFSGSGSGTSYSLTISSMEAEDAATYYCHQYLRSPPTFG
GGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

U.S. patent application Ser. No. 10/724,274 also discloses F200, the Fab′ fragment of M200.

III. Fab′-SH Fragments and Methods for Producing Them

In one preferred embodiment, the proteins of the present invention comprise Fab′-SH fragments of antibodies. Novel methods of producing Fab′-SH fragments are also provided herein. In particular, a starting antibody is digested with either pepsin or papain, either in an immobilized form or preferably in a solution in the presence or absence of a reducing agent, preferably at a pH of about 6.0 to about 8.0, and more preferably about 7.0. The reaction is typically performed at about 15° C. to about 50° C., preferably at about 30° C. to 40° C., and most preferably at about 37° C. Where the starting antibody is an IgG4 type, the digestive enzyme papain is typically preferred. The papain/antibody ratio (weight) is typically from about 1:10 to 1:10⁸, preferably from about 1:10³ to 1:10⁵, and more preferably about 1:10⁴. The digestion is carried out for about 1-100 hours, preferably about 1-10 hours, and more preferably about 3-4 hours. Various reducing agents known in the art are optionally used in the digestion, including, but not limited to, DTT, cysteine, β-mercaptoethylamine, and N-actyl-L-cysteine. Concentrations of the reducing agents are typically about 0.1-100 mM, preferably about 1-50 mM, and more preferably about 1-20 mM.

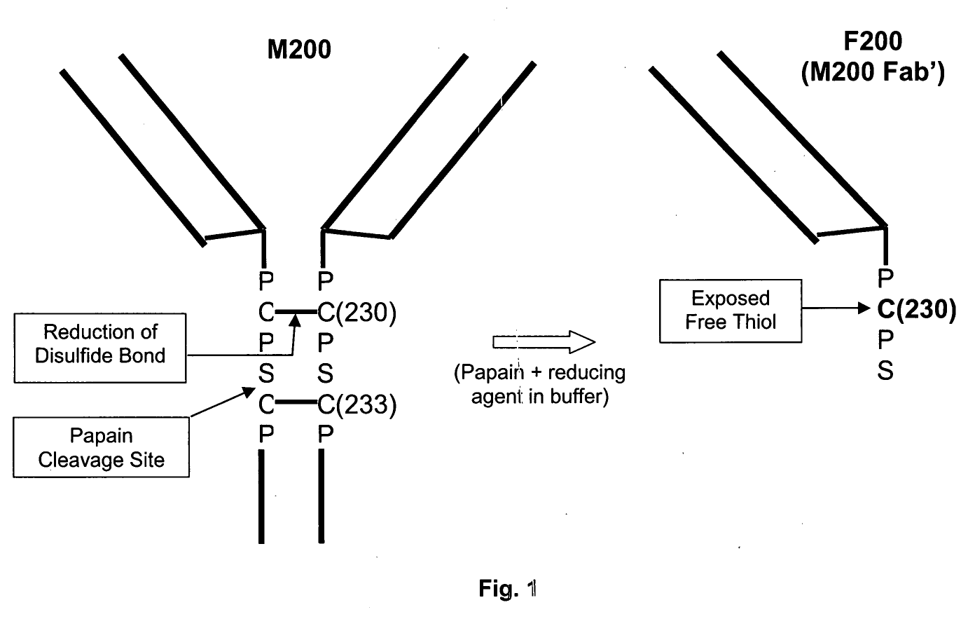

In a preferred embodiment, the starting antibody is an antibody of IgG4 class, preferably a chimeric or humanized IgG4 antibody. In a more preferred embodiment, the antibody is M200 (as provided by SEQ ID NOS: 1-2) or HuMV833. HuMV833 is a humanized anti-VEGF antibody. FIG. 1 provides a schematic depiction of papain digestion of an IgG4 antibody. Papain cleaves between the two intra-heavy chain disulfide bonds. Reduction of the C230-C230 disulfide bond is required for the release of the Fab′ fragment that has an exposed free thiol group at position 230.

Preferably, soluble papain is utilized for digestion processes in the present invention instead of immobilized papain, which is typically used in the art. Immobilized papain often contains sodium azide as a preservative, which is often problematic for clinical manufacturing. The use of soluble papain as disclosed in the present specification avoids this problem. Another advantage of using soluble papain is that antibodies are digested with a low papain/antibody ratio. For example, using soluble papain, with 1:10000 ratio (weight) (e.g., 100 ppm) of papain to antibody, M200 is 99% digested in 3 hours. In contrast, when using immobilized papain, a papain/antibody ratio of 1:5 is required to achieve the same digestion efficiency. Another advantage of using soluble papain is that it is easily removed by cation exchange chromatography (CEX). Further, when soluble papain is stored in sodium azide free preservative at 4-8° C. in dark it loses less than 50% activity in 13 months.

Soluble papain sometimes causes proteolytic digestion of the linkage between protein A and the matrix used in antibody purification, thus releasing protein A into the solution. As a consequence, the methods of the present invention typically further comprise a step of purifying the post digestion mixture before a potential protein A affinity chromatography step. For example, cation exchange chromatography is optionally used to remove papain, the residue reducing agents, undigested starting antibodies, Fc and other impurities. Protein A affinity chromatography is then typically used as an additional subsequent step to remove trace undigested antibodies. The antibody fragments after Protein A purification are optionally subjected to ultrafiltration/diafiltration buffer exchange and formulation, e.g., using methods well known in the art.

The Fab′ fragments, e.g., natalizumab fragments or M200 fragments, produced as described above are in condition to be derivatized with a stabilizing agent as described in more detail below.

IV. Protein Derivative and Methods for Producing Them

The present invention provides compositions comprising stable protein derivatives having at least one thiol group that is coupled to a NAC molecule, NEM molecule or CYS molecule via a disulfide bond. Methods of preparing these stable protein derivatives are also provided. The methods typically comprise coupling the free thiol group of a protein to a molecule such as NAC, NEM or CYS, preferably in the presence of sodium tetrathionate.

In some embodiments, the derivatized proteins comprise antibodies or fragments thereof. In a preferred embodiment, the antibodies bind specifically to integrin molecules, e.g., α5β1 integrin. A preferred antibody is M200, as described above. More preferably, the proteins are Fab′-SH fragments of antibodies, and more preferably of antibodies that bind to integrins, e.g., the Fab′-SH fragment of the M200 antibody which binds to α5β1 integrin or the Fab′-SH fragment of natalizumab which binds to the α4β1 integrin. Methods of making stable derivatives of other antibodies, antibody fragments, e.g., antibody fragments that inhibit angiogenesis, and peptides, e.g., anti-coagulant peptides, are also provided.

The protein derivatives of the present invention are optionally generated by incubating a protein having a free thiol group with NAC, CYS, or NEM for at least about 1 minute, about 5 minutes, about 10 minutes, or about 30 minutes, or about 1 to about 5 hours, and preferably for about 30-60 minutes. The concentration of NAC, or CYS, or NEM is typically about 0.10-100 mM, preferably 1-50 mM, and more preferably 10-40 mM. In some embodiments, the reaction is facilitated by sodium tetrathionate (NTT), which is optionally added into the mixture of the proteins and NAC, CYS, or NEM at a concentration of about 1-100 mM, preferably about 1-50 mM, and more preferably about 10-30 mM and incubated for about 1 minute to several hours, preferably, about 1 minute to 1 hour, and more preferably about 30 minutes, at about 4° C. to 40° C. and preferably at about room temperature, e.g., about 22° C. to about 28° C. The reaction results in the addition of NAC, CYS or NEM to the free thiol group of the protein. In a preferred embodiment, the resulting protein derivatives are further purified and concentrated as described herein.

Where the protein to be derivatized is an antibody, e.g., an IgG4 antibody, a chimeric, or humanized antibody, the starting antibody is typically digested with a papain solution in the presence of NAC. NAC can act as a reducing agent, but is not required for the digestion when soluble papain is used. After digestion, sodium tetrathionate (NaTT) is optionally added to the reaction mixture to react with free thiols, e.g., generated from the reduction of the C230-C230 disulfide bond between the light chain and the heavy chain of M200, thereby forming reactive sulfenylthiosulfate intermediates with which another sulfhydryl, preferably NAC, couples to form a disulfide linkage. The generated molecule, referred to as Fab′NAC, is typically stable in solution, e.g., even in simple phosphate buffer. A preferred Fab′-SH fragment is an Fab′ SH fragment of M200 or any other antibody that inhibits angiogenesis or otherwise directly kills tumor cells. The Fab′NAC produced by the methods of the present invention from an M200 antibody is referred to as F200 Fab′NAC, and has a molecular weight of 48184.4 Daltons (about 48 kD).

One preferred method for producing stable Fab′-SH derivatives comprises the following steps:

1) digesting an antibody, e.g., using a papain solution, preferably in the presence of NAC as described above;

2) producing the Fab′-NAC molecule in the presence of sodium tetrathionate (NaTT);

3) purifying the Fab′Nac molecule, e.g., by cation exchange chromatography (CEX) and protein A chromatography;

4) concentrating the purified Fab′Nac molecules, e.g., using ultrafiltration; and

5) diafiltrating the concentrated Fab′Nac molecules into a formulation buffer.

The stability of the generated protein derivative (such as Fab′NAC) is optionally tested, e.g., via methods known in the art, for example, HPLC or LC-MS (Liquid Chromatography Mass Spectrometry). HPLC is optionally used to evaluate the percent monomer, aggregate and clip formation as a function of time and storage temperature. In the case of antibody having a free thiol, the main degradation pathway is typically dimer formation over time. LC-MS may be used to evaluate the stability of the generated protein derivative (e.g., dimer formation) as a function of time and storage temperature. Typically, Fab′NAC molecules remain as a single homogeneous species and have the predicted molecular weight for a single homogeneous species.

A formulation comprising the compositions of the invention, e.g., a composition comprising a protein or protein derivative, preferably allows the protein or protein derivative to retain its physical, chemical and biological activity over time and at certain temperatures. The formulation is preferably stable for at least about 1 year at refrigerated temperature, e.g., about 2° C. to about 8° C., and about 3 months at room temperature, e.g., about 23° C. to about 27° C. Preferably, a formulation containing the protein derivatives has less than about 5% of protein dimers after one-year storage at refrigerated temperature (about 2-8° C.), or after about 3 months at room temperature (about 23-27° C.) or after about one-month storage at about 37° C. In a preferred embodiment essentially no change in the molecular weight of the generated monomeric protein derivatives is observed after about one-year storage at refrigerated temperature (about 2-8° C.), or after about 3 months at room temperature (about 23-27° C.) or after about one-month storage at about 37° C.

A Fab′NAC or peptide-NAC (peptide stabilized with NAC using the methods provided herein) molecule of the present invention also preferably retains the same binding specificity, e.g., to its antigen, as the parent protein, e.g., antibody. Binding specificity is typically examined via techniques known in the art, including, but not limited to, immunoelectrophoresis, radioimmunoassay, radioimmuno-precipitation, enzyme-linked immuno-sorbent assay (ELISA), dot blot or Western blot assay, and sandwich assays.

The Fab′NACs and peptide-NACs (peptides stabilized using the methods provided herein) of the present invention also preferably retain the same binding affinity, e.g., to an antigen, as the parent protein, e.g., antibody or peptide. The binding affinity of a Fab′NAC or peptide-NAC is optionally determined by Scatchard analysis, by surface plasmon resonance using BIAcore, or by any other method known to those of skill in the art.

In addition to retaining binding specificity and binding affinity, Fab′NACs and peptide-NACs also retain the desired biological activities of their parent proteins. For example, in a preferred embodiment F200 Fab′ NAC inhibits angiogenesis (as does its parent antibody M200) as shown, for example, by its ability to inhibit tube formation in vitro and choroidal neovascularization (CNV) in primate eyes as disclosed in Publication No.: US 2005/0054834 A1, filed Nov. 26, 2003, which is hereby incorporated by reference.

With the same biological specificity and biological activity and the addition of increased stability, e.g., in formulation, the compositions of the present invention provide improved protein pharmaceutical products over what is presently available.

V. Pharmaceutical Formulations

The present invention is also directed to stable liquid and/or lyophilized pharmaceutical formulations of protein compositions comprising one or more free thiol groups and preferably comprising protein derivatives having a thiol group coupled to a molecule such as NAC, CYS or NEM. Preferably, the proteins are antibodies (more preferably of the IgG4 class), antibody fragments, e.g., Fab′ fragments, or peptides, e.g., anti-coagulation peptides, with one or more free thiol groups available for coupling to the molecules described above. In a preferred embodiment, the antibody fragments are Fab′-SH fragments of a chimeric or a humanized antibody, such as an Fab′-SH fragment of M200 or other antibodies that inhibit tumor growth and/or angiogenesis.

The pharmaceutical formulations of the present invention preferably comprise a protein or protein derivative, such as those described immediately above or a mixture thereof dissolved in a pharmaceutically acceptable carrier, preferably an aqueous carrier. A variety of aqueous carriers are optionally used, e.g., water for injection (WFI), or water buffered with phosphate, citrate, acetate, etc., and/or containing salts such as sodium chloride, potassium chloride, etc. The carrier also optionally contains pharmaceutically acceptable excipients such as human serum albumin, polysorbate 80, sugars or amino acids. The formulated proteins or protein derivatives according to the present invention are particularly suitable for parenteral administration, and are optionally administered as an intravenous infusion or by intravitreal, subcutaneous, intramuscular, intravenous, intrathecal, intraventricular, or intrasynovial injection, with intravitreal injection a preferred route of administration. Methods for preparing parenterally administrable formulations are known or apparent to those skilled in the art and are described in more detail in, for example, Remington's Pharmaceutical Science (15^{th} Ed., Mack Publishing Company, Easton, Pa., 1980), which is incorporated herein by reference.

A. Stable Liquid Formulations

In one aspect, the present invention is directed to stable liquid pharmaceutical formulations comprising one or more protein, or protein derivative. Typically, before formulation, the protein is stabilized by coupling a molecule such as an NAC, CYS, or NEM to a free thiol group of the protein, resulting in a stable protein derivative as described above. The generated protein derivatives are stable in the pharmaceutical formulations of the present invention.

The stable liquid formulations of the present invention minimize, for example, denaturation, clipping, or aggregate formation as described above. When the protein or protein derivative is an antibody or antibody fragment or derivative thereof, the formulation aids in maintaining its immunoreactivity, (e.g., ability to bind to an antigen) over time. Preferably, the formulation comprises a sterile, pharmaceutically acceptable liquid formulation containing an antibody, antibody fragment and preferably a derivative thereof as described herein in a buffer having a near neutral pH (pH 5.00-8.00). The protein concentration in the formulation is typically at least about 1, 2, 5, 10, 20, 50 mg/ml, preferably about 1-80 mg/ml and preferably further comprises a buffer of pH 5.00-8.00. Examples of buffers that control the pH in this range include citrate, succinate (such as sodium succinate), histidine, phosphate, and other organic buffers. Citrate (pKa 6.0) is typically a preferred buffer for subcutaneous injection. A preferred buffer comprises about 10-50 mM sodium citrate. Another preferred buffer comprises about 30-70 mM histidine buffer overlaid with N₂.

In some embodiments, the formulation also comprises a surfactant. Exemplary surfactants include, but are not limited to, nonionic surfactants such as polysorbates (e.g. polysorbates 20, 80, such as TWEEN® 20, TWEEN® 80) or poloxamers (e.g. poloxamer 188). The amount of surfactant added is typically such that it aids in reducing aggregation of the protein or protein derivatives and/or minimizes the formation of particulates in the formulation and/or reduces adsorption to the container containing the formulation. The surfactant is typically present in the formulation in an amount from about 0.005% to about 0.5%, preferably from about 0.01% to about 0.1%, more preferably from about 0.01% to about 0.05%, and most preferably from about 0.02% to about 0.04%.

The tonicity of the formulations is also optionally adjusted by adding one or more salts to the formulation. A preferred salt is sodium chloride. MgCl₂, which may protect proteins from deamidation, is also optionally added to the formulation. EDTA, which is commonly used with proteins, formulation is also optionally included in the formulations of the invention.

A preferred formulation of the present invention comprises a buffer comprising sodium citrate at a concentration of about 5-50 mM, preferably about 20-40 mM, and sodium chloride at a concentration of about 80-200 mM, preferably about 80-120 mM.

Exemplary liquid formulations comprise the protein or protein derivative at a concentration of about 20 mg/ml or greater, about 40 mM sodium citrate (pH 6.0) and about 90 mM sodium chloride. Preferred liquid formulations comprise antibodies, antibody fragments, peptides, or derivatives thereof at about 20 mg/ml or greater, about 20-60 mM sodium phosphate (pH 7), about 0.05% Tween 80, and about 75-150 mM NaCl. The formulations also optionally contain free NAC, CYS or NEM, e.g., not coupled to a protein. Preferably the protein is an antibody, an antibody fragment, or a peptide, or more preferably a derivative thereof, and most preferably a Fab′-NAC or peptide-NAC (a peptide coupled to NAC). In a most preferred embodiment, the antibody fragment derivative is F200Fab′-NAC as described herein.

The formulations of the present invention are prepared such that the protein or protein derivative retains its physical, chemical and biological activity. The formulation is preferably stable for at least 1 year at refrigerated temperature, e.g., about 2° C. to about 8° C. and 6 months at room temperature, e.g., about 22° C. to about 28° C.

The analytical methods for evaluating the product stability include methods well known in the art including, but not limited to, UV spectroscopy, size exclusion chromatography (SEC), SDS-PAGE, cation exchange chromatography (CEX), liquid chromatography mass spectrometry (LC/MS), bioanalyzer, HIC, and the like.

B. Stable Lyophilized Formulations

In another aspect, the present invention is directed to stable lyophilized formulations comprising proteins or protein derivatives as described herein. Lyophilization is a freeze-drying process that is often used in the preparation of pharmaceutical products containing an active ingredient to preserve their biological activity. The process generally involves sublimating a previously frozen liquid sample in a vacuum (to remove the ice and/or other frozen solvent), and thereby leaving the non-solvent components intact, in the form of a powdery or cake-like substance. The lyophilized product can be stored for prolonged periods of time, and at elevated temperatures, without loss of biological activity, and can be readily reconstituted into a particle-free solution by the addition of an appropriate diluent. An appropriate diluent is any physiological acceptable liquid in which the lyophilized powder is completely soluble. Water, particularly sterile, pyrogen-free water, is a preferred diluent. The advantage of lyophilization is that the water content is reduced to a level that greatly reduces the various water related molecular events which leads to instability of the protein upon long-term storage. The lyophilized product is also more readily able to withstand the physical stresses of shipping. The reconstituted product is particle free, thus it can be administered without prior filtration.

The following criteria are typically used in developing stable lyophilized protein or protein derivative containing formulations: protein unfolding during lyophilization is preferably minimized; glass transition temperature (Tg) is preferably greater than the product storage temperature; residual moisture is preferably low (about <1% by mass); a preferred shelf life is at least about 3 months, preferably about 6 months, more preferably about 1 year at room temperature, e.g., 22 to 28° C.; a reconstitution time is preferably short, for example, less than about 5 minutes, preferably less than about 2 minutes, and more preferably less than about 1 minute; when the lyophilized product is reconstituted, the reconstituted sample is typically stable for at least about 48 hours, e.g., at about 28° C.

The present stable lyophilized formulations typically comprise a protein or protein derivative and, optionally, free NAC as a stabilizing agent. Adding free NAC to the pre-lyophilized, liquid formulation containing protein or protein derivative helps prevent the formation of the disulfide-linked aggregates in the liquid formulation at about 2-8° C. for a short period of time prior to lyophilization. The protein or protein derivatives are stable in a formulation comprising NAC at a concentration of about 0.1-100 mM, preferably about 1-50 mM, more preferably about 1-5 mM, and most preferably about 1-2.5 mM. The concentration of NAC in the pre-lyophilized liquid formulation is preferably less than about 50 mM, 20 mM, or 5 mM, with a preferred range of about 1 mM to about 2.5 mM.

The protein or protein derivative in the pre-lyophilized liquid formulation is preferably at a concentration of at least about 1, 2, 5, 10, 20, or 50 mg/ml, preferably about 1-10 mg/ml.

A buffer of pH 5.00-8.00, preferably about 6.00, is typically used in the formulation. Examples of buffers that control the pH in this range include, but are not limited to, citrate, succinate (such as sodium succinate), histidine, phosphate, and other organic buffers. A preferred buffer is about 1 -10 mM, and preferably about 5 mM histidine buffer.

A polyol, which acts as a tonicifying agent and a cryoprotector/lyphoprotector, is also optionally included in the lyophilized formulation. In a preferred embodiment, the polyol is a nonreducing sugar, such as sucrose or trehalose, which may also play a role in reducing the reconstitution time of the lyophilized formulation to a particle-free solution. The polyol may be added to the formulation in an amount that typically varies with respect to the desired tonicity of the formulation. Preferably the lyophilized formulation after reconstitution is isotonic, however, hypertonic or hypotonic formulations may also be suitable. Suitable concentrations of the polyol such as sucrose in the pre-lyophilized formulation are in the range from about 100-300 mM, preferably in the range from about 80-200 mM.

The lyophilized formulations of the present invention may also contain a bulking agent such as mannitol that provides good cake properties. Such agents also contribute to the tonicity of the formulations and may provide protection from freeze-thaw stresses and improve long-term stability. A preferred bulking agent is mannitol at a concentration of about 10-55 mM, and preferably about 20-45 mM.

Other tonicity modifiers such as salts (e.g., NaCl, KCl, MgCl₂, CaCl₂, and the like) are optionally added to the pre-lyophilized formulation, e.g., to control osmotic pressure.

Preferred pre-lyophilized formulations typically comprise a solution comprising an IgG type antibody (preferably an IgG4 type antibody, and more preferably a chimeric or humanized IgG4 antibody) or fragment thereof or a peptide at about 10 mg/ml or greater, about 5 mM histidine (pH 6.0), about 0.005-0.03% polysorbate 20 or 80, and about 80-130 mM sucrose, and 10-55 mM mannitol. A preferred antibody fragment is a Fab′-SH fragment. The above pre-lyophilized formulation is lyophilized to form a dry, stable powder, which is easily reconstituted to a particle-free solution suitable for administering to humans. Preferably, samples are kept frozen for about3 hours at about −40° C. before initiating the primary drying cycle. A preferred primary drying cycle is carried out at about −20° C. at a pressure of about 150 mTorr for about 10 hours. A preferred secondary drying cycle is carried out at about 20° C. at a pressure of about 150 mTorr for about 8 hours.

Where the protein is an antibody or antibody fragment or a derivative thereof or a biologically active peptide, a lyophilized formulation stabilizes biological activity (e.g., binding specificity and binding affinity) of the antibody or peptide, and prevents the protein, e.g., intended for administration to human subjects, from becoming physically and chemically degraded, e.g., in the final product.

VI. Diagnostic and Therapeutic Applications

The proteins and protein derivatives, e.g., stabilized proteins, of the present invention are optionally used for various therapeutic and non-therapeutic purposes. Where the protein, or protein derivatives are antibodies or antibody fragments or derivatives thereof (e.g., Fab′-NAC), they are optionally used as affinity purification agents. They are also useful in diagnostic assays, such as detecting expression of an antigen of interest in specific cells, tissues, or serum. For diagnostic applications, the protein or derivatives typically will be labeled with a detectable moiety, including radioisotopes, fluorescent labels, and various enzyme substrate labels. The derivatives are also optionally employed in any known assay method, such as competitive binding assays, direct and indirect sandwich assays, and immunoprecipitation assays. The derivatives are also useful for in vivo diagnostic assays. Generally, the derivatives are labeled with a radionucleotide when used in this fashion, so that the antigen or cell expressing it can be localized using immunoscintigraphy.

Kits can also be supplied for use with the derivatives in the protection against or detection of a cellular activity or for the presence of a selected cell surface receptor or the diagnosis of disease. The derivatives, which may be conjugated to a label or toxin, or unconjugated, are included in the kits with buffers, such as Tris, phosphate, carbonate, etc., stabilizers, biocides, inert proteins, e.g., serum albumin, or the like, and a set of instructions for use. Generally, these materials will be present in less than about 5% wt. based on the amount of active antibody, and usually present in total amount of at least about 0.001% wt. based again on the antibody concentration. Frequently, it will be desirable to include an inert extender or excipient to dilute the active ingredients, where the excipient may be present in from about 1 to 99% wt. of the total composition. Where a second antibody capable of binding to the modified antibody is employed in an assay, this is usually present in a separate vial. The second antibody is typically conjugated to a label and formulated in an analogous manner with the antibody derivatives described above.

The pharmaceutical formulations of the present invention have various therapeutic applications. The formulations are optionally used to treat a patient suffering from, or predisposed to, diseases or disorders, including, but not limited to, cancer, inflammatory conditions, such as asthma or inflammatory bowel diseases, autoimmune diseases, coronary artery diseases, heart failure, multiple sclerosis, infectious diseases, and the like.

The types of cancer that are optionally treated include, but are not limited to, breast cancer, squamous cell cancer, small cell lung cancer, non-small cell lung cancer, gastrointestinal cancer, pancreatic cancer, glioblastoma, cervical cancer, ovarian cancer, bladder cancer, hepatoma, colon cancer, colorectal cancer, endometrial carcinoma, salivary gland carcinoma, kidney cancer, liver cancer, prostate cancer, vulval cancer, thyroid cancer, hepatic carcinoma, melanoma, hematopoietic cancers, such as leukemias, lymphomas and myelomas, and various types of head and neck cancer. Autoimmune diseases that may be treated with the formulations of the present invention include, but are not limited to, Addison's disease, autoimmune diseases of the ear, autoimmune diseases of the eye such as uveitis, autoimmune hepatitis, inflammatory bowel disease, Crohn's disease, diabetes (Type I), epididymitis, glomerulonephritis, Graves' disease, Guillain-Barre syndrome, Hashimoto's disease, hemolytic anemia, systemic lupus erythematosus, multiple sclerosis, myasthenia gravis, pemphigus vulgaris, osteoporosis, psoriasis, rheumatoid arthritis, sarcoidosis, scleroderma, Sjogren's syndrome, spondyloarthropathies, thyroiditis ulcerative colitis, vasculitis, and the like.

For example, tumor growth depends on vascularization. Angiogenesis (i.e. the growth of new blood vessels) within a tumor begins when release of one or more of the pro-angiogenic growth factor(s) (e.g., FGF, VEGF, PDGF, etc) locally activates the endothelial cells. These activated endothelial cells then develop new blood vessels by binding to the fibronectin in the extracellular matrix, e.g., via α5β1 integrin receptors. The integrin α5β1 is upregulated in tumor neovasculature and its ligand, fibronectin, is enriched in malignant basement epithelium. Molecules that block the interaction between α5β1 and fibronectin are known to inhibit tumor angiogenesis in vitro and in vivo, as do agents that impede the angiogenic properties of VEGF. Tumor metastasis depends on the ability of endothelial and cancer cells to migrate to and invade target tissues. Integrins are essential for cell migration and invasion as they bind directly to the components of the extracellular matrix. Integrin α5β1 which binds specifically to fibronectin is up-regulated on blood vessels in human tumor biopsies. M200 and F200 are potent inhibitors of the α5β1 receptor and thereby inhibit the angiogenesis and cell migration processes that promote tumor growth, metastasis, and the various autoimmune and inflammatory disorders that involve angiogenesis and vascularization.

In addition, M200 and F200 show efficacy in in vivo models of choroidal neovascularization (in monkey eyes) and macular degeneration (in rabbit eyes), as disclosed in the U.S. patent application with Publication No.: US 2005/0054834 A1, and U.S. Ser. No. 10/830,956, filed Apr. 23, 2004, each of which is incorporated by reference in its entirety. Thus, the formulations of the present invention are optionally used as therapeutics for ophthalmic disorders that affect the retina, lens and/or cornea of the mammalian eye, particularly disorders involving modulation of vascularization or wound healing. Among the most important retinal disorders are macular holes and degeneration (particularly age-related macular degeneration), choroidal neovascularization, sub-retinal neovascularization, retinal tears and lesions (particularly of the RPE), acute retinal necrosis syndrome (ARN), traumatic chorioretinopathies or contusion (Purtscher's Retinopathy), disorders associated with retinal edema and ischemia (e.g. retinal vasculitis and occlusion associated with Eales disease and systemic lupus erythematosus), uveitis and diabetic retinopathy. The most important disorders of the lens are cataracts, which may be associated with metabolic diseases or drug side effects, and refractive errors. Among the most important disorders of the cornea are those related to corneal defects, including corneal ulcers, wounds and scarring related to corneal surgery (e.g. laser surgery or corneal transplantation), and the consequences of dry eye and/or Sjogren's syndrome.

The binding of α5β1 integrin to fibronectin has been established as part of a cell adhesion process. Thus, stable F200 formulations of the present invention are optionally used in the study, diagnosis, treatment or prevention of diseases and conditions which relate to cell adhesion, including, but not limited to: arthritis, asthma, allergies, adult respiratory distress syndrome, cardiovascular disease, thrombosis or harmful platelet aggregation, allograft rejection, neoplastic disease, psoriasis, multiple sclerosis, CNS inflammation, Crohn's disease, ulcerative colitis, glomerular nephritis and related inflammatory renal disease, diabetes, ocular inflammation (such as uveitis), atherosclerosis, inflammatory and autoimmune diseases.

The formulations are administered by any suitable means, including parenteral subcutaneous, intraperitoneal, intrapulmonary, and intranasal, intravitreal, intrathecal, intraventricular, or intrasynovial and if desired for local immunosuppressive treatment, intralesional administration. Parenteral infusions include intramuscular, intravenous, intraarterial, intraperitoneal, or subcutaneous administration. In addition, the protein or protein derivatives are suitably administered by pulse infusion, particularly with declining doses of derivatives.

The formulations are optionally administered for prophylactic and/or therapeutic treatments. In therapeutic application, the formulations are administered to a patient already affected by the particular disease, in an amount sufficient to cure or at least partially arrest the condition and its complications. An amount adequate to accomplish this is defined as a “therapeutically effective dose.” Amounts effective for this use will depend upon the severity of the condition and the general state of the patient's own immune system, but generally range from about 0.0001 to about 100 mg/kg of the therapeutic protein per dose, with dosages of about 1 to 10 mg per patient being more commonly used.

In prophylactic applications, the formulations are administered to a patient not already in a disease state to enhance the patient's resistance to the disease. Such an amount is defined to be a “prophylactically effective dose.” In this use, the precise amounts again depend upon the patient's state of health and general level of immunity, but generally range from about 0.1 to 100 mg per dose, especially dosages of about 1 to 10 mg per patient.

Single or multiple administrations of the formulations are optionally carried out with dose levels and pattern being selected by the treating physician. In any event, the pharmaceutical formulations should provide a quantity of the proteins or the derivatives of this invention sufficient to effectively treat the patient.

Where the therapeutic agent in the formulation is an antibody against α5β1 integrin or a Fab′-SH fragment of the antibody (e.g. F200) or a derivative of the antibody and/or the Fab′-SH fragment, the present invention provides for methods for measuring efficacy in modulating angiogenesis, for example, in an animal model. These methods allow screening of formulations comprising derivatives of a Fab′ of an antibody against α5β1 integrin according to the present invention to determine safe, effective therapeutic dosages.

Pathological conditions (e.g., injury or tumor growth) that involve neovascularization events are susceptible to treatment using the formulations of the present invention. Tumors characterized, in part, by angiogenesis are particularly susceptible to treatment using the proteins or protein derivatives of the present invention and more preferably the Fab′-NAC molecules of the present invention. A tumor can be benign, for example, a hemangioma, teratoma, and the like, or can be malignant, for example, a carcinoma, sarcoma, glioblastoma, astrocytoma, neuroblastoma, retinoblastoma, and the like. Malignant tumors that are diagnosed using a method of the invention include, for example, carcinomas such as lung cancer, breast cancer, prostate cancer, cervical cancer, pancreatic cancer, colon cancer and ovarian cancer; glioblastoma; and sarcomas such as osteosarcoma and Kaposi's sarcoma, provided the tumor is characterized, at least in part, by angiogenesis associated with α5β1 expression by the newly forming blood vessels. The present invention also provides methods for testing the formulations of the present invention, using tissue and animal model systems. In a preferred embodiment, the tissue may be injured to create lesions and to promote choroidal neovascularization. Alternatively, the animal or tissue may be exposed to any of a variety of means to induce tumor formation such as exposure to carcinogenic chemicals or ionization radiation. Injury may be accomplished by any suitable means, including mechanical, chemical, or biological means. Exemplary mechanical means of injury include cutting, piercing or clamping. Chemical means include applying agents to the tissue that cause necrosis, apoptosis, or loss of cell to cell contact. Biological means include treatment with infectious agents, such as viruses, bacteria or prions. A preferred method of creating lesions is through the use of a laser. Any laser capable of injuring the tissue is optionally used, with CO₂ gas lasers being a preferred type, a most preferred type being a OcuLight GL (532 nm) Laser Photo-coagulator with a IRIS Medical® Portable Slit Lamp Adaptor. Other laser sources are also suitable provided they can produce laser light from about 300 to about 700 mwatts, and lesions less than 200 μm, preferably less than 100 μm, more preferably from about 50 to about 100 μm in diameter, and most preferably about 75 to 25 μm in diameter. Typically the laser light is applied to the tissue for a fraction of a second. Normally less than about 0.5 second, more preferably less than about 0.1 second, most preferably less than about 0.05 second.

The formulations, of the present invention, e.g., formulations comprising Fab′ fragments of antibodies or derivatives thereof against an integrin, e.g., α5β1 integrin, are optionally administered directly into the region to be treated, for example, directly into a neoplastic tumor, to the eye via eye drops or intravitreal injection, where the pathological condition involves the eye; or intrasynovially, where the condition involves a joint.

Monitoring of clinically relevant progress is another aspect of the present invention. Monitoring a target tissue is carried out by any suitable method known in the art. Preferred methods include microscopy, nuclear magnetic resonance, X-ray, and the like. In the case of eye tissue, indirect ophthalmoscopic examination of the posterior chamber of the eye, and biomicroscopic examination of the anterior segment of the eye are typically used. A preferred method of monitoring the extent of choroidal neovascularization is by intravenously injecting a fluorescein dye, and examining the target tissue by fluorescein angiography.

A preferred method of screening the effectiveness of Fab′-SH fragments or derivatives thereof of anti-α5β1 integrin antibodies such as those described herein in inhibiting or preventing neoangiogenesis is by creating lesions in the retina of an animal, applying the derivatives to the lesions, and then monitoring the progression of neoangiogenesis in the damaged tissue relative to suitable control experiments.

The Fab′ fragment derivatives that bind α5β1 -integrin of the present invention are useful in reducing or inhibiting angiogenesis associated with α5β1 integrin expression, or a pharmaceutical formulation containing a Fab′-SH fragment or derivatives thereof, is optionally used for treating any pathological condition that is characterized, at least in part, by angiogenesis

Angiogenesis associated with α5β1 integrin expression can occur locally, for example, in the retina of an individual suffering from diabetic retinopathy, or can occur more systemically, for example, in an individual suffering from rheumatoid arthritis or a malignant neoplasm. Since regions of angiogenesis can be localized or can be more systemically dispersed, one skilled in the art would select a particular route and method of administration of the therapeutic antibodies, antibody fragments or derivatives thereof of the present invention based, in part, on this factor.

For example, in an individual suffering from diabetic retinopathy, where angiogenesis associated with α5β1 integrin expression is localized to the retina, the anti-α5β1 integrin antibody, antibody fragment or derivative thereof is formulated in a pharmaceutical formulation convenient for use as eye drops or intravitreal injection which can be administered directly to the eye. In comparison, in an individual suffering from a metastatic carcinoma, the agent in a pharmaceutical composition is formulated so that is administered intravenously, orally or by another method that distributes the agent systemically. Thus, the formulations of the present invention are optionally administered by various routes, including, but not limited to, intravenously, orally, or directly into the region to be treated, for example, directly into a neoplastic tumor; via eye drops or intravitreal injection where the pathological condition involves the eye; or intrasynovially, where the condition involves a joint; or intrathecally or intraventricularly when the pathological condition involves the central nervous system.

A preferred method of administering the formulations of the present invention is by way of injection, either intradermally, intravenously or directly into the joint or tissue which is involved in a pathological condition. For example, when retinal tissue has been damaged or is otherwise in a pathological state, formulations of the present invention are injected intravitreally into an affected eye. In one embodiment of the present invention administration of the formulation to one eye leads to clinically beneficial effects in both eyes (assuming both eyes are injured or diseased). It appears that newly formed blood vessels are “leaky,” allowing antibodies, antibody fragments or derivatives that applied to the first eye to pass into the blood stream where they are transported to the second eye. When applied to the eye in this manner, the dose is preferably less than 5 μM, more preferably between about 0.5 and 2 μM, and most preferably between about 0.1 and 1.0 μM. Where indicated, treatment takes the form of multiple doses, given over an area or period of time. Dosages in a multidose format may all be identical, or independently determined and applied. This result has also led to an additional method of treating lesions with associated neoangiogenesis comprising systemic application of an effective amount of the therapeutic formulations of the present invention (for example by intravenous injection) wherein neoangiogenesis of an injured tissue is inhibited or prevented.

Other antibodies and peptides that are useful in treating disease are also optionally used in pharmaceutical formulations as described above. For additional antibody and peptide therapeutics, see, e.g., U.S. Pat. Nos. 6,475,488, 6,528,481, 6,423,313, 6,239,101, 6,902,522, and 6,841,354.

### EXAMPLES

The following examples are provided by way of illustration only and not by way of limitation. For example, not all antibodies and peptides are illustrated in the examples, but the same methods apply to any protein, peptide or antibody with a free thiol group. Those of skill in the art will readily recognize a variety of non-critical parameters that are optionally modified to yield essentially the same or similar results.

### Example 1

### Generation of Fab′SH Antibody Fragments by Papain Digestion.

A chimeric anti-α5β1 integrin antibody, M200, (described in the U.S. patent application with Publication No.: US 2005/0054834 A1, filed Nov. 26, 2003, which is incorporated herein by reference in its entirety), or humanized anti-VEGF IgG4 antibody (HuMV833-PDL) (both IgG4 antibodies) were buffer exchanged into 20 mM sodium phosphate and 20 mM N-acetyl-L-cysteine at a pH of 7.0. Soluble papain enzyme in an enzyme/antibody ratio of 1:10000 was added. The mixture was rotated at 37° C. for 3 hours. After digestion, the mixture was purified to remove Fc fragments and undigested IgG leaving a purified Fab′-SH antibody fragment. Liquid Chromatography Mass Spectrometry (LC-MS) analysis revealed that the main cleavage sites of HuMV833 and M200 are identical. The main cleavage site for HuMV833 is between S226 and C227. The corresponding cleavage site for M200 is between S232 and C233, which when cleaved gives rise to a Fab′-SH fragment (FIG. 1) having a free thiol group. These results indicated that the papain cleaves between two disfulfide bonds of an IgG4 antibody regardless of the composition of the complementarity determining regions (CDRs) and gives rise to a molecule containing a free thiol.

### Example 2

### Production of Stable Fab′ Derivatives of M200

Fab′ derivatives were produced by three major steps, including digestion, chemical treatment after digestion, and formulation. Various conditions were tested for each step to develop the optimal ways of making the stable formulation of the derivatives, including the type of reducing agents, the type of treatment after digestion, and the type of formulation. Three separate matrices containing different combinations of experimental conditions were designed and the experiments carried out as described below. Table 1 summarizes the conditions and results of Matrix #3, which was representative of two other experimental matrices.

The general experimental procedure was as follows: the antibody M200 [SEQ ID NOS: 1 and 2] was buffer exchanged into 20 mM sodium phosphate at pH 7.0; soluble papain enzyme was added in an enzyme/antibody ratio of 1:10000; a reducing agent was added into the reaction mixture at a selected concentration (according to column labeled “Digestion Reducing Agent” in Table 1) which included NAC, CYS, NEM, β-MEA (β-mercaptoethylamine) or dithiothreitol (DTT). The mixture was rotated at 37° C. for 3-4 hours. After digestion, a chemical treatment agent, for example, sodium tetrathionate (NaTT), which facilitated the chemical reaction, e.g., the addition of NAC or NEM or CYS to a free thiol, was added at the indicated concentration (see Table 1) and incubated for 30 minutes at room temperature. This preparation was then buffer exchanged into a formulation solution, for example, a solution comprising 20 mM sodium phosphate, 100 mM sodium chloride at pH 7.4 (PBS) with or without NAC (see Table 1).

Additional downstream steps including cation exchange chromatography (CEX) and protein A chromatography for purification and ultrafiltration for concentration; and diafiltration into the formulation buffer were carried out according to well-known methods in order to obtain a purified F200 Fab′NAC in the desired formulation.

The stability of the protein derivatives was analyzed by LC-MS or HPLC after several days in the formulation. A lower percentage of Fab′ dimer measured in the resulting formulation was indicative of higher stability. As shown in Table 1, the lower percentage of Fab′ dimer measured in the formulations for Fab′-NAC (F200 Fab′-NAC) and Fab′-NEM (F200 Fab′-NEM) indicated higher stability as compared to other derivatives. There were less than 2% of Fab′ dimers when the derivatives were prepared using conditions 1, 2 and 8 after 8 days. HPLC analysis showed that F200Fab′NAC molecules generated using condition 8 were stable 8 days in the formulation at 4° C., 25° C. and 37° C., with less than 5% dimers present in the formulation. Using LC-MS, the F200Fab′NAC molecules were shown to be a single species having the predicted molecular weight of 48184.4 Daltons.

TABLE 1
Matrix #3 Conditions and Results

| | | | | | % | % | % | % |
|---|---|---|---|---|---|---|---|---|
| | Digestion | | Chemical | Formulation | Dimer | Dimer | Dimer | Dimer |
| | Reducing Agent | | Treatment | Solution | Day 0 | Day 4 | Day 6 | Day 8 |
| 1 | 1 | mM DTT | No Treatment | NAC | 0.5 | 0.6 | 0.6 | 1.2 |
| 2 | 1 | mM DTT | NEM block | PBS | 0.2 | 0.7 | 0.7 | 0.9 |
| 3 | 1 | mM DTT | NaTT + NEM | PBS | 6.4 | 14 | 15 | 17 |
| 4 | 20 | mM Cys | NEM block | PBS | 0.7 | 4.7 | 6.1 | 7.4 |
| 5 | 20 | mM Cys | NaTT | PBS | 0.8 | 11 | 20 | 23 |
| 6 | 20 | mM Cys | NaTT + NEM | PBS | 0.9 | 11 | 20 | 24 |
| 7 | 20 | mM β-MEA | NATT | PBS | 0.7 | 8.4 | 10 | 11 |
| 8 | 20 | mM NAC | NATT | PBS | 0.7 | 1.0 | 1.6 | 1.8 |
| 9 | 20 | mM NAC | No Treatment | PBS | 1.8 | 16 | 20 | 24.0 |

### Example 3

### Stability Studies of Formulations Comprising F200Fab′NAC

F200Fab′NAC molecules prepared as described above were stored at a concentration of 20 mg/ml in a formulation comprising 40 mM sodium citrate, 90 mM sodium chloride, 0.05% Tween 80 at pH 6.0.

Size Exclusion Chromatography (SEC) was used to examine the stability of F200Fab′NAC at 5° C., 25° C., and 37° C., for up to three months in the formulation. Data corresponding to the percentage of F200Fab′NAC dimer, and percentage of clip formation was measured over a period of 12 weeks (at time points 0, 1, 2, 4, 8 and 12 weeks) and at 5° C., 25° C., and 37° C. respectively.

Over a 12-week period, minimal changes were observed in the percentage dimer levels of the samples stored at 5° C. (i.e. less than 1% dimer observed). The samples at 25° C. and 37° C. had percentage dimer levels of 2.57% and 7.23%, respectively at 12 weeks. The percentage of F200Fab′NAC monomer measured indicated more than 98% in the formulation at 5° C. over a period of three months in the formulation. Similarly, the percentage monomer was 97.91 and 92.61 after 12 weeks at 25° C. and 37° C., respectively. Furthermore, very low percentages (e.g. 0.10-0.25%) of clips (i.e. F200Fab′NAC proteins of less than full molecular weight) were observed for the formulations at 5° C., 25° C., and 37° C. and increased only minimally over the 12-week period. These data suggest that the formulation is sufficiently stable to have a shelf life of about 1 year at a storage temperature of about5° C.

Cation Exchange Chromatography (CEX) profiles were also measured and used to determine the percentage of F200Fab′NAC monomers over a period of 3 months of storage at 5° C., 25° C., and 37° C., respectively. At 5° C., only very minimal changes were observed in the isoform distribution of the F200Fab′NAC monomer peak profile at ˜15.5 minutes. For samples incubated at 25 and 37° C., a degradant peak was observed to grow at ˜26.3 minutes. The degradant levels increased as a function of temperature and time. It is possible that this peak corresponds to the dimer component observed on SEC.

The stability of F200Fab′NAC was also evaluated by reducing and non-reducing SDS-PAGE. After 3 months in the formulation at 5° C., 25° C., and 37° C., respectively, samples containing F200Fab′NAC were run in duplicate on reducing and non-reducing SDS-PAGE gels. Non-reduced SDS-PAGE gave rise to an increase in the aggregate band at ˜100 KD as a function of temperature. These results are consistent with the SEC measurements described above that also show an increase in aggregation at elevated temperatures. The mass of the aggregate band corresponds to ˜100 KD, which suggests that the aggregate formed is a dimer. Free light chain contaminant in the sample was observed in the non-reducing gels. The reduced gel primarily showed a single band corresponding to the mass of the light chain band. Because the masses of the light chain and the heavy chain fragment were very close, both components were most likely co-eluting.

LC-MS studies were also carried out and further confirmed the stability of F200Fab′NAC at in the formulation over three months at 5° C., 25° C., and 37° C. The observed LC/MS spectral profiles and molecular weight data indicated that the product was fairly homogeneous and was composed of Fab′ blocked with a single NAC molecule. Low levels of Fab′ blocked with 2 NAC molecules were also observed as was the presence of free light chain in the molecule. After six months, LC-MS indicated only minimal changes in the mass of Fab′-232-NAC (F200Fab′NAC) molecule as a function of temperature and time. These data strongly suggest that forming a bond between the free thiol and NAC molecule stabilizes the Fab′-SH against aggregation.

In addition, the binding potency of F200Fab′NAC to fibronectin was examined via comparative ELISA assay relative to M200 binding to fibronectin. The data, gathered over a 12 week period for samples stored at 5° C., 25° C. and 37° C., indicated that F200Fab′NAC retained a binding specificity and affinity to fibronectin that is comparable to M200 throughout the 12 week study.

In summary, the data demonstrated that the Fab′NAC derivative is significantly more stable than the underivatized F200Fab′ fragment. Even at the low concentration of 2 mg/mL, F200Fab′ (without NAC derivatization) exhibited significant aggregation at 25 and 37° C. in less than 2 weeks. In addition, increased aggregate formation was observed at 5° C. In contrast, the Fab′NAC derivative exhibited minimal changes in aggregation levels at the concentration of 20 mg/mL at 5° C., and was observed to be considerably more stable at the elevated temperatures of 25° and 37° C.

### Example 4

### Stability Study of the Formulation Comprising F200Fab′CYS

F200Fab′CYS was generated by following the same procedure as used in producing F200Fab′NAC except that CYS was used instead of NAC. The Fab′ fragment of M200 (5.0 mg/ml) was dialyzed into PBS with 5 mM cysteine. An amount of 100 mM NaTT was added to the PBS solution and the solution was incubated at room temperature for about 30 minutes. The post-reaction mixture was dialyzed with a PBS solution. The stability of the F200-Fab′ Cys derivative was monitored using size exclusion chromatography (SEC) (as described above) over a period of 4 weeks at 5° C. and 25° C. Minimal change in the percentage of dimer (˜1% or less change) was observed over 4 weeks period at both 5° C. and 25° C. in samples containing F200Fab′CYS or F200Fab′NAC. The data indicated that, F200Fab′CYS is stable in a phosphate buffer saline formulation for up to at least 1 month at 5° C. and 25° C.

### Example 5

### Stability Studies of Lyophilized Formulation of F200Fab′NAC.

Pre-lyophilization liquid formulations were prepared comprising 10 mg/ml F200Fab′NAC, 1 mM to 5 mM N-acetyl-L-cysteine, 5 mM histidine, 90 mM sucrose, 40 mM mannitol, and 0.005% Tween 80. The liquid preparation was then frozen and lyophilized. The lyophilized formulations were reconstituted with half the fill volume resulting in a post-lyophilization concentration of approximately 20 mg/mL. LC-MS and HPLC were used to detect the percentage of dimer and aggregation after reconstitution. The data indicated that there was minimal aggregation (i.e. less than 0.10 to about 0.36%) at 1.0 to 2.5 mM concentrations of added NAC. Minimal changes in percentage of aggregation were observed with 1 mM NAC at both 5° C. and 25° C. up to two weeks post reconstitution, indicating that F200Fab′NAC stabilized the lyophilized formulation. Similar results were observed when the formulation comprised 2.5 mM or 5 mM NAC.

In order to examine whether the F200Fab′NAC is stable in a liquid formulation before lyophilization, stability of above liquid formulation was monitored at 5° C. over a period of 36 days. After 36 days, more than 95% of monomers were observed in the formulation at 5° C., indicating that the formulation is fairly stable pre-lyophilization.

### Example 6

### Binding Specificity of F200Fab′NAC

In order to determine whether or not F200Fab′NAC retains the binding specificity of its parent antibody M200, the tissue distribution of the M200 and F200Fab′NAC was examined in rabbit eyes as described below.

Twenty four animals were divided into two groups with twelve rabbits in each group. For Group 1, animals were dosed with ¹²⁵I-F200Fab′(NAC), by bolus intravitreal injection of 50 μl/eye (100 μg containing 10 μCi) to both eyes of each animal by a veterinary ophthalmologist. For Group 2, animals were injected with a bolus of ¹²⁵I-M200, by intravitreal injection of 50 μl/eye (300 μg containing 10 μCi) into each eye of each animal by a veterinary ophthalmologist. Prior to administration, animals were anesthetized with an intramuscular (IM) injection of xylazine (5 mg/kg) followed by an IM injection of ketamine (25 mg/kg). The eyes were prepared by rinsing with 1% Betadine® ophthalmic solution. The eyes were then be rinsed with a 0.9% sterile saline solution. A topical anesthetic was instilled in each eye before dose administration. A topical antibiotic was instilled in each eye following dose administration.

Tissue samples from the injected animals were analyzed for radioactivity using solid scintillation counting (SSC). Terminal blood samples were analyzed for radioactivity. Serial serum samples were subdivided into aliquots for radioanalysis, trichloroacetic acid (TCA) precipitation, and ELISA. Terminal blood was centrifuged to obtain the buffy coat and plasma. The buffy coat and plasma were analyzed for radioactivity. Vitreous humor samples were obtained and were subdivided into aliquots for radioanalysis, TCA precipitation, and ELISA. All blood, plasma, serum, and vitreous humor samples were analyzed in duplicate if sample size allowed. All thyroid and ocular tissues, with the exception of vitreous humor, were analyzed as single samples. All samples were counted for at least 5 minutes.

Tissue distribution of F200Fab′NAC and M200 was examined at 4 hours, and at 1, 4, 7, 14, and 21 days after injection. Two animals were typically sacrificed at each time point. Four eyes were evaluated per time point.

Over the tested period of 504 hours (3 weeks), the measured tissue distribution of F200Fab′NAC was similar to that of M200 in various locations of the eye, including cornea, aqueous humor, lens, vitreous humor, vitreous humor wipe, retina, RPE, choroid, and sclera. Further, the temporal distribution in eye tissue was also similar. For example, for both M200 and F200Fab′NAC, the concentration in vitreous humor peaked at 4 hours before decreasing, whereas in RPE both peaked at 24 hours before decreasing.

In addition, no undesired crystalline deposit or any evidence-of inflammation was observed after the injection. These data demonstrate that F200Fab′NAC was well-tolerated and is able to reach the back of the eye after intravitreal injection in tested rabbits.

### Example 7

### Efficacy of F200Fab′-NAC in a Rabbit Model of Advanced Macular Degeneration (AMD)

A hydron pellet based sustained-release system for both VEGF and bFGF has been shown to produce florid irreversible retinal neovascularization (NV) in the rabbit after intravitreal implantation (See, e.g., Wong et al., “Intravitreal VEGF and bFGF produce florid neovascularisation and hemorrhage in the rabbit,” Current Eye Research 22: 140-147 (2001)) and to produce choroidal neovascularization (CNV)following suprachoroidal implantation (See e.g., Carvalho et al., “Stimulation of choroidal neovascularization in the rabbit through sustained release of VEGF and bFGF, ” Poster presentation at “Fifth Annual Vision Research Conference, April 2001” Satellite Symposium of ARVO, Fort Lauderdale, Fla.)

Choroidal neovascularization (CNV) is the hallmark of exudative advanced macular degeneration (AMD). Thus, CNV induced by intravitreal VEGF pellets in rabbits represents a good whole animal model for testing the efficacy of AMD therapeutics.

F200 Fab′NAC and M200 were shown to inhibit CNV in this rabbit model as assessed by fundus photograph scoring of degree of hemorrhage, and leakage of fluorescein determined by fluorescein angiography (FA) according to the following method (also disclosed in U.S. patent application Ser. No. 10/830,956, filed Apr. 23, 2004). In adult male and female Dutch belted rabbits (N=50), a limited conjunctival peritomy was made in the superotemporal quadrant, followed by a 4 mm full thickness scleral incision concentric to and 3 mm posterior to the limbus. Care was taken not to incise through the choroid. A hydron implant containing 20 μg each of VEGF and bFGF (Wong et al., “Intravitreal VEGF and bFGF produce florid neovascularisation and hemorrhage in the rabbit,” Current Eye Research 22: 140-147 (2001)) was placed as posterior as possible to rest in the suprachoroidal space, which was created by passing a cyclodialysis spatula between the choroid and sclera.

Intravitreal injections of M200 (600 mg) and F200Fab′NAC (200 mg) in citrate buffer were made 2 mm posterior to the limbus with a 30-gauge needle at both time of implant (day 0) and day 15. Intravenous (I.V.) M200 (10 mg/kg) was administered at day 0 and day 15. Fundus photographs, OCT, and fluorescein angiographs (FAs) were taken at 1, 2, 3, 4, and 8 weeks later.

Clinical grading of fundus photographs and FAs were performed by two masked graders on a scale of 0, 1 (mild), 2 (moderate), 3 (moderately severe), and 4 (severe). Generally, increased hemorrhaging as indicated by areas of deeper and/or darker redness in the fundus photographs results in increased scores. The clinical grading scores for the images are included beside each fundus photograph. Animals were enucleated at week 4 (N=40) and week 8 (N=10) for histology.

The VEGF/bFGF hydron implants produced a robust, persistent model with high penetrance and yielded 75% of rabbits with CNV. In this robust rabbit model of CNV, 5 of 8 (62.5%) of implanted control eyes developed CNV by week 4.

Treatment with M200 or F200Fab′NAC resulted in significant inhibition of sub-retinal hemorrhaging due to the VEGF/bFGF implant. The clinical grading of the fundus photographs taken over the course of the treatment period revealed significant inhibition of subretinal hemorrhage for treatment groups compared to placebo. For intravitreal M200, p=0.130, 0.03, 0.003, 0.001 for weeks 1-4 respectively. For intravitreal F200Fab′NAC, p=0.042, 0.004, 0.002, 0 for weeks 1-4. For intravenous M200, p=0.009, 0.001, 0.005, 0 for weeks 1-4. Grading of the FA images also showed trends toward inhibition of CNV. Interestingly, the parent mAb, M200, showed significant inhibition of CNV when administered by I.V. route, but intravitreal M200 was less efficacious than F200Fab′NAC.

Although the foregoing invention has been described in some detail by way of illustration and example for clarity and understanding, the description is not intended to limit the invention. It will be readily apparent to one of ordinary skill in the art in light of the teachings of this invention that certain changes and modifications may be made thereto without departing from the spirit and scope of the appended claims. For example, all the techniques and compositions described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A composition comprising a protein, the protein comprising a thiol group coupled to N-acetyl-L-cysteine, N-ethyl-maleimide, or cysteine.

2. The compositions of claim 1, wherein said protein comprises an antibody, an antibody fragment, or a peptide.

3. The composition of claim 2, wherein said antibody fragment comprises a Fab′ fragment.

4. The composition of claim 3, wherein the Fab′ fragment comprises a Fab′ fragment of an IgG1, IgG2, IgG3, or IgG4 antibody.

5. The composition of claim 2, wherein the antibody or antibody fragment binds to an integrin.

6. The composition of claim 2, wherein the antibody, antibody fragment or peptide inhibits angiogensis.

7. The composition of claim 5, wherein the integrin comprises α5β1, αvβ3, or α4β1 integrin.

8. The composition of claim 2, wherein said antibody comprises a heavy chain amino acid sequence of SEQ ID NO: 1 and a light chain amino acid of SEQ ID NO: 2.

9. The composition of claim 2, wherein said antibody fragment comprises a Fab′ fragment of an antibody comprising a heavy chain amino acid sequence of SEQ ID NO: 1 and a light chain amino acid of SEQ ID NO: 2.

10. The composition of claim 2, wherein the peptide comprises an anti-coagulation peptide.

11. The composition of claim 1, wherein the protein comprises trastuzumab, omalizumab, efalizumab, bevacizumab, daclizumab, palivizumab, natalizumab, gemtuzumab, ozogamicin, eptifibatide, abciximab, alemtuzumab, cetuximab, infliximab, rituximab, basiliximab, palivizumab, epratuzumab, apolizumab, labetuzumab, human B-type natriuretic peptide, nesiritide, or urodilatin.

12. A liquid or lyophilized formulation comprising the composition of claim 1.

13. A stable liquid pharmaceutical formulation comprising a protein and a pharmaceutically acceptable carrier, said protein comprising a thiol group coupled to N-acetyl-L-cysteine, N-ethyl-maleimide, or cysteine.

14. The pharmaceutical formulation of claim 13, wherein said protein comprises an antibody, an antibody fragment, or a peptide.

15. The pharmaceutical formulation of claim 14, wherein said antibody fragment compises a Fab′ fragment.

16. The pharmaceutical formulation of claim 15, wherein the Fab′ fragment comprises a Fab′ fragment of an IgG1, IgG2, IgG3, or IgG4 antibody.

17. The pharmaceutical formulation of claim 14, wherein the antibody or antibody fragment binds to an integrin.

18. The pharmaceutical formulation of claim 17, wherein the integrin comprises α5β1, αvβ3, or α4β1 integrin.

19. The pharmaceutical formulation of claim 14, wherein said antibody comprises a heavy chain amino acid sequence of SEQ ID NO: 1 and a light chain amino acid of SEQ ID NO: 2.

20. The pharmaceutical formulation of claim 14, wherein said antibody fragment comprises a Fab′ fragment of an antibody comprising a heavy chain amino acid sequence of SEQ ID NO: 1 and a light chain amino acid of SEQ ID NO: 2.

21. The pharmaceutical formulation of claim 14, wherein the peptide comprises an anti-coagulation peptide.

22. The pharmaceutical formulation of claim 13, wherein the protein comprises trastuzumab, omalizumab, efalizumab, bevacizumab, daclizumab, palivizumab, natalizumab, gemtuzumab, ozogamicin, eptifibatide, abciximab, alemtuzumab, cetuximab, infliximab, rituximab, basiliximab, palivizumab, epratuzumab, apolizumab, labetuzuma, human B-type natriuretic peptide, nesiritide, or urodilatin.

23. A stable lyophilized pharmaceutical formulation comprising a protein, said protein comprising a thiol group coupled to N-acetyl-L-cysteine, N-ethyl-maleimide, or cysteine.

24. The pharmaceutical formulation of claim 23, wherein said protein comprises an antibody, an antibody fragment, or a peptide.

25. The pharmaceutical formulation of claim 24, wherein said antibody fragment comprises a Fab′ fragment.

26. The pharmaceutical formulation of claim 25, wherein the Fab′ fragment comprises a Fab′ fragment of an IgG1, IgG2, IgG3, or IgG4 antibody.

27. The pharmaceutical formulation of claim 24, wherein the antibody or antibody fragment binds to an integrin.

28. The pharmaceutical formulation of claim 27, wherein the integrin comprises α5β1, αvβ3, or α4β1 integrin.

29. The pharmaceutical formulation of claim 24, wherein said antibody comprises a heavy chain amino acid sequence of SEQ ID NO: 1 and a light chain amino acid of SEQ ID NO: 2.

30. The pharmaceutical formulation of claim 24, wherein said antibody fragment comprises a Fab′ fragment of the antibody comprising a heavy chain amino acid sequence of SEQ ID NO: 1 and a light chain amino acid of SEQ ID NO: 2.

31. The pharmaceutical formulation of claim 24, wherein the peptide comprises an anti-coagulation peptide.

32. The pharmaceutical formulation of claim 23, wherein the protein comprises trastuzumab, omalizumab, efalizumab, bevacizumab, daclizumab, palivizumab, natalizumab, gemtuzumab, ozogamicin, eptifibatide, abciximab, alemtuzumab, cetuximab, infliximab, rituximab, basiliximab, palivizumab, epratuzumab, apolizumab, labetuzuma, human B-type natriuretic peptide, nesiritide, or urodilatin.

33. A method for preparing a composition, the method comprising:
incubating a protein with a stabilizing agent in the presence of sodium tetrathionate, wherein the protein comprises a free thiol and the stabilizing agent comprises N-acetyl-L-cysteine, N-ethyl-maleimide, or cysteine, thereby coupling said stabilizing agent to the thiol group of the protein.

34. The method of claim 33, wherein said protein comprises an antibody, an antibody fragment, or a peptide.

35. The method of claim 34, wherein said antibody fragment comprises a Fab′ fragment.

36. The method of claim 35, wherein the Fab′ fragment comprises a Fab′ fragment of an IgG1, IgG2, IgG3, or IgG4 antibody.

37. The method of claim 34, wherein the antibody or antibody fragment binds to an integrin.

38. The method of claim 35, wherein the integrin comprises α5β1, αvβ3, or α4β1 integrin.

39. The method of claim 34, wherein said antibody comprises a heavy chain amino acid sequence of SEQ ID NO: 1 and a light chain amino acid of SEQ ID NO: 2.

40. The method of claim 34, wherein said antibody fragment comprises a Fab′ fragment of the antibody comprising a heavy chain amino acid sequence of SEQ ID NO: 1 and a light chain amino acid of SEQ ID NO: 2.

41. The method of claim 34, wherein the peptide comprises an anti-coagulation peptide.

42. The method of claim 33, wherein the protein comprises trastuzumab, omalizumab, efalizumab, bevacizumab, daclizumab, palivizumab, natalizumab, gemtuzumab, ozogamicin, eptifibatide, abciximab, alemtuzumab, cetuximab, infliximab, rituximab, basiliximab, palivizumab, epratuzumab, apolizumab, labetuzuma, human B-type natriuretic peptide, nesiritide, or urodilatin.

43. A method of coupling a Fab′ fragment of an antibody to N-acetyl-L-cysteine, the method comprising:
a) digesting the antibody with papain, thereby producing the Fab′ fragment of said antibody, wherein the Fab′ fragment comprises a free thiol group;
b) incubating said Fab′ fragment with N-acetyl-cysteine in the presence of sodium tetrathionate, thereby coupling said N-acetyl-cysteine to said Fab′ fragment via the free thiol group.

44. The method of claim 43, further comprising purifying said Fab′ fragment.

45. The method of claim 44, wherein said Fab′ fragment binds to an integrin.

46. The method of claim 45, wherein the integrin comprises α5β1, αvβ3, or α4β1 integrin.

47. The method of claim 43, wherein said antibody comprises the amino acid sequence having a heavy chain amino acid sequence of SEQ ID NO: 1 and a light chain amino acid of SEQ ID NO: 2.

48. The method of claim 43, wherein the Fab′ fragment comprises a Fab′ fragment of an IgG1, IgG2, IgG3, or IgG4 antibody.
